SEQUENCE LISTING (11 sequences)

SEQ ID NO: 1 - protein, 124 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 2 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, Artificial Sequence
  Description of Artificial Sequence Synthetic peptide
SYGMH

SEQ ID NO: 4 - protein, 17 aa, Artificial Sequence
  Description of Artificial Sequence Synthetic peptide
VIWFDGTKKYYTDSVKG

SEQ ID NO: 5 - protein, 15 aa, Artificial Sequence
  Description of Artificial Sequence Synthetic peptide
DRGIGARRGPYYMDV

SEQ ID NO: 6 - protein, 11 aa, Artificial Sequence
  Description of Artificial Sequence Synthetic peptide
RASQSISSYLN

SEQ ID NO: 7 - protein, 7 aa, Artificial Sequence
  Description of Artificial Sequence Synthetic peptide
AASSLQS

SEQ ID NO: 8 - protein, 9 aa, Artificial Sequence
  Description of Artificial Sequence Synthetic peptide
QQSYSTPLT

SEQ ID NO: 9 - protein, 10 aa, Artificial Sequence
  Description of Artificial Sequence Synthetic peptide
GFAFSSYGMH

SEQ ID NO: 10 - protein, 453 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0270830 A1
(43) Pub. Date: Aug. 15, 2024

(54) METHODS FOR TREATING ALZHEIMER'S DISEASE

(71) Applicant: Biogen MA Inc., Cambridge, MA (US)

(72) Inventors: Karen Smirnakis, Weston, MA (US); Patrick Ryland Burkett, Cambridge, MA (US); Kimberly Umans, Waltham, MA (US)

(21) Appl. No.: 18/567,674

(22) PCT Filed: Jun. 6, 2022
(86) PCT No.: PCT/US22/32397

§ 371 Date: Dec. 6, 2023

Related U.S. Application Data
(60) Provisional application No. 63/335,186, filed on Apr. 26, 2022.
(60) Provisional application No. 63/326,369, filed on Apr. 1, 2022.
(60) Provisional application No. 63/219,319, filed on Jul. 7, 2021.
(60) Provisional application No. 63/197,949, filed on Jun. 7, 2021.

(51) Int. Cl. C07K 16/18; A61K 39/00; A61P 25/28

(52) U.S. Cl. CPC C07K 16/18; A61P 25/28; A61K 2039/505; A61K 2039/545

(57) ABSTRACT

Provided are methods for treating Alzheimer's disease in a human subject in need thereof when the subject develops an Amyloid Related Imaging Abnormality (ARIA) or a hypersensitivity reaction during a treatment regimen comprising administration of multiple doses of an anti-beta-amyloid antibody to the subject.

### CROSS-REFERENCE TO RELATED APPLICATIONS

This application claims priority to U.S. Provisional Application Nos. 63/197,949, filed on Jun. 7, 2021, 63/219,319, filed on Jul. 7, 2021, 63/326,369 filed on Apr. 1, 2022, and 63/335,186 filed on Apr. 26, 2022, the entirety of each of which is incorporated herein by reference.

### TECHNICAL FIELD

This disclosure relates generally to methods for treating Alzheimer's disease.

### BACKGROUND

Alzheimer's disease (AD) is a progressive neurodegenerative disorder clinically characterized by cognitive impairment, behavioral disturbances, psychiatric symptoms, and disability in activities of daily living. These clinical manifestations are characteristic of AD.

AD International estimates that the number of people living with dementia worldwide will increase from the current value of 47 million to 131 million by 2050. Being the most common cause of dementia, AD accounts for 60 to 80% of dementia cases. In the United States, it is estimated that 5.2 million Americans suffer from dementia caused by AD, and that by 2050 the prevalence will double or triple unless an effective treatment is found.

Clinical research criteria for dementia due to AD have been recently updated and conforming to the current concept of the disease, a diagnostic framework was developed to embrace pre-dementia stages of AD (e.g., prodromal AD). The main neuropathological hallmarks of the disease are (i) extracellular senile (neuritic) plaques containing aggregated β-amyloid (Aβ) peptides and (ii) intraneuronal neurofibrillary tangles (NFTs) composed of abnormal hyperphosphorylated Tau protein. The “amyloid cascade” hypothesis proposes that the driving force behind the disease process is the accumulation of Aβ resulting from an imbalance between Aβ production and Aβ clearance in the brain.

Aβ is a peptide generated from the metabolism of amyloid precursor protein. Several Aβ peptide alloforms exist (e.g., Aβ40, Aβ42). These monomeric peptides have a variable tendency to aggregate into higher order dimers and oligomers. Through a process of fibrillogenesis, soluble oligomers may transition into insoluble deposits having a β pleated sheet structure. These deposits are also referred to as amyloid plaques and are composed of predominantly fibrillar amyloid. Both soluble and fibrillar forms of Aβ appear to contribute to the disease process.

Biomarker, clinicopathologic, and cohort studies suggest that the disease process commences 10 to 20 years before the clinical onset of symptoms, and some of the early pathological findings include the deposition of neocortical neuritic plaques and mesial temporal NFTs followed years later by neocortical NFTs.

Thus, there is a need for methods to treat Alzheimer's disease patients.

### SUMMARY

This disclosure fulfills the need for methods to treat Alzheimer's disease patients. The current disclosure describes, among other things, methods of treating Alzheimer's disease that include administering an anti-beta-amyloid antibody. The present disclosure encompasses a recognition of certain risks associated with methods of treating Alzheimer's disease with an anti-beta-amyloid antibody, including, e.g., Amyloid Related Imaging Abnormality (ARIA) and/or a hypersensitivity reaction. In some embodiments, the present disclosure provides particular methods that mitigate such risks to improve safety in treatment of patients with Alzheimer's disease.

In some embodiments, the disclosure provides methods for treating Alzheimer's disease in a human subject in need thereof by administering to the human subject multiple doses of an anti-beta-amyloid antibody, and monitoring for ARIA by obtaining brain magnetic resonance imaging (MRI) during treatment with the anti-beta-amyloid antibody. In some embodiments, ARIA is detected in the human subject (e.g., by MRI).

In some embodiments, the ARIA detected in the human subject is radiographically mild ARIA-E with mild clinical symptoms or no clinical symptoms, and administering of the antibody is continued. In some embodiments, the ARIA detected in the human subject is radiographically mild ARIA-H with no clinical symptoms, and administering of the antibody is continued.

In some embodiments, the ARIA detected in the human subject is radiographically mild ARIA-E with clinical symptoms (e.g., moderate or severe clinical symptoms) or radiographically moderate or severe ARIA-E. In some embodiments, the administering of the antibody is suspended until radiographic resolution. In some embodiments, the administering is suspended until clinical symptoms, if any, resolve. Alternatively, in some embodiments, the administering of the antibody is suspended until radiographic stabilization and clinical symptoms, if any, resolve.

In some embodiments, the ARIA detected in the human subject is radiographically mild ARIA-H with clinical symptoms or radiographically moderate or severe ARIA-H. In some embodiments, the administering of the antibody is suspended until radiographic stabilization. In some embodiments, the administering is suspended until clinical symptoms, if any, resolve. Alternatively, in some embodiments, the administering of the antibody is suspended until radiographic resolution and clinical symptoms, if any, resolve.

In some embodiments, provided methods include monitoring for ARIA by MRI at particular time points. In some embodiments, an MRI is obtained and/or performed prior to the first dose of 6 mg/kg. In some embodiments, an MRI is obtained and/or performed prior to the first dose of 10 mg/kg. In some embodiments, an MRI is obtained and/or performed prior to the third dose of 10 mg/kg. In some embodiments, an MRI is obtained and/or performed prior to the sixth dose of 10 mg/kg.

In some embodiments, an MRI is obtained and/or performed prior to the first dose of 10 mg/kg and prior to the sixth dose of 10 mg/kg. In some embodiments, an MRI is obtained and/or performed at each of: (i) prior to the first dose of 6 mg/kg, (ii) prior to the first dose of 10 mg/kg, (iii) prior to the third dose of 10 mg/kg, and (iv) prior to the sixth dose of 10 mg/kg.

In some embodiments, the disclosure provides methods for treating Alzheimer's disease in a human subject in need thereof by:
administering to the human subject multiple doses of an anti-beta-amyloid antibody, wherein the human subject develops Amyloid Related Imaging Abnormality-H (ARIA-H) (e.g., as detected by brain magnetic resonance imaging (MRI)) during treatment with the anti-beta-amyloid antibody, wherein the ARIA-H comprises ten or more new incident microhemorrhages or greater than two focal areas of superficial siderosis, and
administering further doses of the anti-beta-amyloid antibody to the human subject, at a dose level not reduced compared to the last dose administered prior to the detection of ARIA-H, only after a clinical evaluation and a follow-up brain MRI demonstrates radiographic stabilization as indicated by no increase in size or number of ARIA-H.

In some embodiments, the present disclosure provides methods that include:
administering to a population of human subject multiple doses of an anti-beta-amyloid antibody, and
suspending administration in those human subjects that develop Amyloid Related Imaging Abnormality-H (ARIA-H) during treatment that is radiographically (i) mild ARIA-H with symptoms or (ii) moderate or severe ARIA-H with or without symptoms.

In some embodiments, the method further comprises resuming administration when a follow-up MRI demonstrates radiographic stabilization and/or any symptoms resolve.

In some embodiments, a subject with ARIA-H also experiences seizure, such as, e.g., status epilepticus.

In some embodiments, the method further comprises continuing administration in those subjects that do not develop ARIA-H, or develop radiographically mild ARIA-H without symptoms.

In some embodiments, the present disclosure provides methods that include:
administering to a population of human subject multiple doses of an anti-beta-amyloid antibody; and
suspending administration in those human subjects that develop Amyloid Related Imaging Abnormality-E (ARIA-E) during treatment that is radiographically moderate or severe ARIA-E.

In some embodiments, the method further comprises resuming administration when a follow-up MRI demonstrates radiographic resolution. In some embodiments, the method further comprises resuming administration when a follow-up MRI demonstrates radiographic resolution and clinical symptoms, if present, have resolved.

In some embodiments, the present disclosure provides methods that include:
administering to a population of human subject multiple doses of an anti-beta-amyloid antibody; and
suspending administration in those human subjects that develop radiographically mild Amyloid Related Imaging Abnormality-E (ARIA-E) during treatment that have moderate or severe clinical symptoms.

In some embodiments, the method further comprises resuming administration when a follow-up MRI demonstrates radiographic resolution and clinical symptoms have resolved.

In some embodiments, a subject with ARIA-E also experiences seizure, such as, e.g., status epilepticus.

In some embodiments, the method further comprises continuing administration in those subjects that do not develop ARIA-E, or develop radiographically mild ARIA-E without symptoms or mild ARIA-E with mild symptoms.

In some embodiments, the present disclosure provides methods that include:
(i) administering multiple doses of an anti-beta-amyloid antibody to a population of human subjects with Alzheimer's disease, and
(ii) in those subjects that develop ARIA (e.g., as detected by brain MRI) during treatment with the anti-beta-amyloid antibody:
   (a) performing a follow-up brain MRI; and if the MRI demonstrates radiographic stabilization and/or resolution;
   (b) administering further doses of the anti-beta-amyloid antibody to the human subjects, at a dose level not reduced compared to the last dose administered prior to the detection of ARIA.

In some embodiments, the present disclosure provides methods that include:
(i) administering multiple doses of an anti-beta-amyloid antibody to a population of human subjects with Alzheimer's disease, and
(ii) in those subjects that develop ARIA-H (e.g., as detected by brain MRI) during treatment with the anti-beta-amyloid antibody:
   (a) performing a follow-up brain MRI; and if the MRI demonstrates radiographic stabilization as indicated by no increase in size or number of ARIA-H;
   (b) administering further doses of the anti-beta-amyloid antibody to the human subjects, at a dose level not reduced compared to the last dose administered prior to the detection of ARIA-H.

In some embodiments, the present disclosure provides methods that include:
(i) administering multiple doses of an anti-beta-amyloid antibody to a population of human subjects with Alzheimer's disease, and
(ii) in those subjects that develop intracerebral hemorrhage that is greater than 1 cm in diameter during treatment with anti-amyloid-beta antibody: permanently discontinuing administration of anti-beta-amyloid antibody.

Alternatively, in some embodiments, the present disclosure provides methods that include:
(i) administering multiple doses of an anti-beta-amyloid antibody to a population of human subjects with Alzheimer's disease, and
(ii) in those subjects that develop intracerebral hemorrhage that is greater than 1 cm in diameter during treatment with anti-amyloid-beta antibody: suspending dosing until MRI demonstrates radiographic stabilization and symptoms, if present, resolve.

In some embodiments, the present disclosure encompasses a recognition that management of ARIA does not differ between those subjects that are ApoE ε4 carriers and ApoE ε4 non-carriers.

In some embodiments, the multiple doses of the anti-beta-amyloid antibody are administered intravenously to the human subject(s).

In some embodiments, the multiple doses of the anti-beta-amyloid antibody are administered to the human subject(s) at intervals of once every four weeks.

In some embodiments, the multiple doses of the anti-beta-amyloid antibody comprise administering at least two doses of the antibody in an amount of 6 mg/kg of body weight of the human subject(s).

In some embodiments, the multiple doses of the anti-beta-amyloid antibody comprise administering at least two doses of the antibody in an amount of 10 mg/kg of body weight of the human subject(s).

In some embodiments, the multiple doses of the anti-beta-amyloid antibody comprise administering at least two doses of the antibody in an amount of 6 mg/kg of body weight of the human subject(s) administered intravenously once every four weeks.

In some embodiments, the multiple doses of the anti-beta-amyloid antibody comprise administering at least two doses of the antibody in an amount of 10 mg/kg of body weight of the human subject(s) administered intravenously once every four weeks.

In some embodiments, provided methods are performed on human subject(s) who have a confirmed amyloid beta pathology. In some embodiments, amyloid beta pathology is confirmed prior to administering the antibody.

In some embodiments, provided methods are performed on human subject(s) who have received a brain MRI within one year prior to administering the antibody.

In some embodiments, the multiple doses of the anti-beta-amyloid antibody comprise administering at least twelve doses of the antibody.

In some embodiments, provided methods include performing a brain MRI on the human subject prior to the fifth, seventh, ninth, and/or twelfth dose of the antibody. In some embodiments, provided methods include performing a brain MRI on the human subject prior to the seventh and twelfth doses of the antibody. In some embodiments, provided methods include performing a brain MRI on the human subject prior to each of the fifth, seventh, ninth, and twelfth doses of the antibody.

In some embodiments, provided methods include monitoring for ARIA by MRI at particular time points. In some embodiments, an MRI is obtained and/or performed prior to the first dose of 6 mg/kg. In some embodiments, an MRI is obtained and/or performed prior to the first dose of 10 mg/kg. In some embodiments, an MRI is obtained and/or performed prior to the third dose of 10 mg/kg. In some embodiments, an MRI is obtained and/or performed prior to the sixth dose of 10 mg/kg.

In some embodiments, an MRI is obtained and/or performed prior to the first dose of 10 mg/kg and prior to the sixth dose of 10 mg/kg. In some embodiments, an MRI is obtained and/or performed at each of: (i) prior to the first dose of 6 mg/kg, (ii) prior to the first dose of 10 mg/kg, (iii) prior to the third dose of 10 mg/kg, and (iv) prior to the sixth dose of 10 mg/kg.

In some embodiments, administering the anti-beta-amyloid antibody can cause amyloid related imaging abnormalities-edema (ARIA-E) and -hemosiderin deposition (ARIA-H). In some embodiments, the present disclosure encompasses a recognition that management of ARIA does not differ between those subjects that are ApoE ε4 carriers and ApoE ε4 non-carriers.

In some embodiments, provided methods are performed on human subject(s) who concurrently receive aspirin in daily doses of 325 mg or less. The present disclosure encompasses a recognition that subject(s) concurrently receiving aspirin in daily doses of 325 mg or less did not have an increased risk of ARIA or intracerebral hemorrhage.

In some embodiments, provided methods are performed on human subject(s) who concurrently receive aspirin in doses greater than 325 mg, other antiplatelet drugs or anticoagulants.

In some embodiments, the disclosure provides methods for treating Alzheimer's disease in a human subject in need thereof that include:
administering a first dose of an anti-beta-amyloid antibody to the human subject in an amount of 1 mg antibody/kg of body weight of the human subject, followed by a second dose in an amount of 1 mg antibody/kg of body weight of the human subject four weeks after the first dose;
in four week intervals after the second dose, administering doses 3 and 4 to the human subject in an amount of 3 mg antibody/kg of body weight of the human subject;
in four week intervals after administration of dose 4, administering doses 5 and 6 of the antibody to the human subject in an amount of 6 mg antibody/kg of body weight of the human subject;
in four week intervals after administration of dose 6, administering doses 7, 8, 9, 10, 11, and 12 of the antibody to the human subject in an amount of 10 mg antibody/kg of body weight of the human subject; and
in four week intervals after administration of dose 12, administering maintenance doses of the antibody to the human subject in an amount of 10 mg antibody/kg of body weight of the human subject.

In some embodiments, provided methods are performed on a human subject who has a confirmed amyloid beta pathology. In some embodiments, amyloid beta pathology is confirmed prior to administering the antibody.

In some embodiments, a baseline brain MRI has been obtained for the human subject prior to the administering of the anti-beta-amyloid antibody. In some embodiments, a brain MRI was performed on the human subject within one year prior to administering dose 1 of the antibody. In some embodiments, provided methods further comprise performing a brain MRI on the human subject within one year prior to administering dose 1 of the antibody.

In some embodiments, a brain MRI is performed on the human subject after administering dose 4 of the antibody and prior to administering dose 5 of the antibody.

In some embodiments, a brain MRI is performed on the human subject after administering dose 6 of the antibody and prior to administering dose 7 of the antibody.

In some embodiments, a brain MRI is performed on the human subject after administering dose 8 of the antibody and prior to administering dose 9 of the antibody.

In some embodiments, a brain MRI is performed on the human subject after administering dose 11 of the antibody and prior to administering dose 12 of the antibody.

In some embodiments, a brain MRI is performed on the human subject after administering dose 6 of the antibody and prior to administering dose 7 of the antibody, and after administering dose 11 of the antibody and prior to administering dose 12 of the antibody.

In some embodiments, a brain MRI is performed on the human subject at each of the following time points: (i) after administering dose 4 of the antibody and prior to administering dose 5 of the antibody, (ii) after administering dose 6 of the antibody and prior to administering dose 7 of the antibody, (iii) after administering dose 8 of the antibody and prior to administering dose 9 of the antibody, and (iv) after administering dose 11 of the antibody and prior to administering dose 12 of the antibody.

In some embodiments, administering the anti-beta-amyloid antibody can cause amyloid related imaging abnormalities-edema (ARIA-E) and -hemosiderin deposition (ARIA-H).

In some embodiments, the human subject develops ARIA-H during treatment with the anti-beta-amyloid antibody, wherein the ARIA-H is radiographically (i) mild ARIA-H with symptoms or (ii) moderate or severe ARIA-H with or without symptoms, and the method further comprises suspending treatment until a follow-up MRI demonstrates radiographic stabilization and/or any symptoms resolve.

In some embodiments, the human subject develops ARIA-H as detected by brain MRI during treatment with the anti-beta-amyloid antibody, wherein the ARIA-H comprises ten or more new incident microhemorrhages or greater than two focal areas of superficial siderosis, and the method comprises administering further doses of the anti-beta-amyloid antibody to the human subject, at a dose level not reduced compared to the last dose administered prior to the detection of ARIA-H, only after a clinical evaluation and a follow-up brain MRI demonstrates radiographic stabilization as indicated by no increase in size or number of ARIA-H.

In some embodiments, the ARIA-H is detected by the brain MRI performed on the human subject after administering dose 6 of the antibody and prior to administering dose 7 of the antibody.

In some embodiments, the ARIA-H is detected by the brain MRI performed on the human subject after administering dose 11 of the antibody and prior to administering dose 12 of the antibody.

In some embodiments, the disclosure provides methods for treating Alzheimer's disease in a human subject in need thereof that include:
administering to the human subject multiple doses of an anti-beta-amyloid antibody; and
monitoring the human subject for ARIA during the first 8 doses of an anti-beta-amyloid antibody.

In some embodiments, the ARIA is ARIA-H.

In some embodiments, the ARIA-H is detected by a brain MRI performed on the human subject after administering dose 6 of the antibody and prior to administering dose 7 of the antibody.

In some embodiments, the method further comprises administering further doses of the anti-beta-amyloid antibody to the human subject after a follow up MRI demonstrates radiographic stabilization.

In some embodiments, the amount of antibody administered in each of the further doses is the same as or greater than the amount of antibody administered in the last dose administered prior to detection of ARIA-H.

In some embodiments, the disclosure provides methods that include:
administering to a human subject multiple doses of an anti-beta-amyloid antibody, wherein the human subject develops a hypersensitivity reaction; and discontinuing treatment upon the first observation of any signs or symptoms consistent with a hypersensitivity reaction.

In some embodiments, the human subject has Alzheimer's disease. In some embodiments, the human subject has a confirmed amyloid beta pathology.

In some embodiments, the hypersensitivity reaction comprises angioedema, urticaria, or angioedema and urticaria.

In some embodiments, provided methods further comprise administering a therapeutic agent for treating the hypersensitivity reaction.

In some embodiments, the present disclosure provides methods that include:
(i) administering multiple doses of an anti-beta-amyloid antibody to a population of human subjects with Alzheimer's disease, and
(ii) in those human subjections that develop a hypersensitivity reaction, discontinuing administration of the anti-beta-amyloid antibody and administering a therapeutic agent for treating the hypersensitivity reaction, where the hypersensitivity reaction is or comprises angioedema and/or urticarial.

In some embodiments of any of the methods described herein, the anti-beta-amyloid antibody comprises a heavy chain variable region (VH) and a light chain variable region (VL), wherein the VH comprises a first complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

In some embodiments of any of the methods described herein, the anti-beta-amyloid antibody comprises a VH and a VL, wherein the VH comprises the amino acid sequence of SEQ ID NO:1 and the VL comprises the amino acid sequence of SEQ ID NO:2.

In some embodiments of any of the methods described herein, the anti-beta-amyloid antibody comprises a heavy chain comprising the amino acid sequence of SEQ ID NO:10 and a light chain comprising the amino acid sequence of SEQ ID NO:11.

In some embodiments of any of the methods described herein, the anti-beta-amyloid antibody is administered by intravenous infusion. In some embodiments, administration of the anti-beta-amyloid antibody by intravenous infusion is over approximately one hour. In some embodiments, administration of the anti-beta-amyloid antibody by intravenous infusion is performed via a 0.2 or 0.22 micron in-line filter.

In some embodiments of any of the methods described herein, the Alzheimer's disease is mild Alzheimer's disease, early Alzheimer's disease, prodromal Alzheimer's disease, mild Alzheimer's disease dementia, mild cognitive impairment due to Alzheimer's disease, mid-stage Alzheimer's disease, or late-stage Alzheimer's disease.

In some embodiments, any of the provided methods for treating Alzheimer's disease can be used to treat patients with mild cognitive impairment or mild dementia stage of disease. In some embodiments, treatment using provided methods described herein are initiated in a Alzheimer's disease patient with mild cognitive impairment or mild dementia stage of disease.

In some embodiments of any of the methods described herein, the method further entails measuring the levels of p-tau and/or t-tau in the CSF of the human subject prior to, during, and/or post treatment.

In some embodiments of any of the methods described herein, the anti-beta-amyloid antibody is formulated as a pharmaceutical composition that comprises: anti-beta-amyloid antibody at a concentration of 50 mg/ml to 250 mg/ml; methionine at a concentration of 5 mM to 150 mM; Arg·HCl at a concentration of 50 mM to 200 mM; histidine at a concentration of 10 mM to 30 mM; PS80 at a concentration of 0.01% to 0.1%; and sucrose at a concentration of 0 to 3%. In some embodiments, the pharmaceutical composition further comprises a thiol-containing antioxidant at a concentration of 0.02 mM to 4 mM. In some embodiments, the pharmaceutical composition has a pH of 5.2 to 6.2. In some embodiments, the pharmaceutical composition has a pH of 5.2 to 6.0. In some embodiments, the pharmaceutical composition has a pH of 5.3 to 5.7. In some embodiments, the pharmaceutical composition has a pH of 5.5. In some embodiments, the thiol-containing antioxidant is selected from the group consisting of GSH, GSSG, the combination of GSH and GSSG, cystine, cysteine, and the combination of cysteine and cystine. In some embodiments, the thiol-containing antioxidant is GSH, GSSG, or a combination of GSH and GSSG.

In some embodiments of any of the methods described herein, the anti-beta-amyloid antibody is formulated at a concentration of 100 mg/ml in a pharmaceutical composition comprising:
Arg·HCl at a concentration of 150 mM;
methionine at a concentration of 10 mM;
histidine at a concentration of 20 mM; and
PS80 at a concentration of 0.05%,
wherein the composition has a pH of 5.5.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### DETAILED DESCRIPTION

### Alzheimer's Disease

Alzheimer's disease, abbreviated herein as AD, is a dementia that is primarily identified by clinical diagnosis and established by markers of the disease.

AD is a continuum having certain operationally defined stages of disease progression. AD pathology begins prior to the onset of clinical symptoms. For example, amyloid plaques, one marker of AD pathology, form 10-20 years prior to the onset of AD dementia. The currently recognized stages of AD include preclinical, prodromal, mild, moderate, and severe. These stages may be further divided into subcategories based on the severity of symptoms and measures of AD progression.

Because AD does not occur in discrete stages, those skilled in the art will recognize that the differences between patient groups may not be distinct in a particular clinical setting. Nevertheless, the clinical disease stage can be characterized by measures, and changes in these measures over time, such as Aβ accumulation (CSF/PET), synaptic dysfunction (FDG-PET/fMRI), tau-mediated neuronal injury (CSF), brain structure (volumetric MRI), cognition, and clinical function. (Jack C R, et al. Hypothetical model of dynamic biomarkers of the Alzheimer's pathological cascade. Lancet Neurol., 2010; 9(1):119-28).

In some embodiments, amyloid-beta is a marker to establish AD. In some embodiments, AD is confirmed by amyloid-beta (Aβ) pathology.

Current core clinical criteria for all dementia, referred to as the NINCDS-ADRDA criteria (McKhann G M, V. diagnosis of dementia due to Alzheimer's disease: Recommendations from the National Inst. on Aging-Alzheimer's Association workgroups on diagnostic guidelines for Alzheimer's disease. Alzheimer's & Dementia, 7 (2011) 263-269), are known in the art and can be employed in practicing this invention. They include cognitive or behavioral impairment involving impaired ability to acquire and remember new information, impaired reasoning and handling of complex tasks, impaired visuospatial abilities, impaired language functions (speaking, reading, writing), and changes in personality, behavior, or comportment. Alzheimer's disease is currently diagnosed using the core criteria and is typically characterized by symptoms which have a gradual onset over months to years, not sudden over hours or days (insidious onset). There is usually a clear-cut history of worsening of cognition by report or observation in Alzheimer's disease subjects.

Other diagnostic classification systems have evolved as new information on AD has become available. These systems include the International Working Group (IWG) new research criteria for diagnosis of AD (Dubois B et al., Lancet Neurol., 2007; 6(8):734-736), IWG research criteria, (Dubois et al., Lancet Neurol., 2010; 9(11):1118-27), NIA/AA Criteria (Jack C R et al. Alzheimer's Dement., 2011; 7(3):257-62), and DSM-5 criteria (American Psychiatric Association, DSM-5, 2013). These classification systems can also be employed in diagnosing AD subjects for treatment according to the methods of this disclosure.

### Patients

The term “patient” is meant to include any human subject for whom diagnosis, prognosis, prevention, or therapy for Alzheimer's disease is desired, and includes a human subject in need of treatment. Those in need of treatment include those already with AD, as well as those prone to have AD, or those in which the manifestation of AD is to be prevented. Typical patients will be men or women aged 40 to 90 (e.g., 45 to 90, 50 to 90, 55 to 90, 60 to 90). In some embodiments, the disclosure provides a method of treating a patient with AD (including, without limitation, patients with preclinical, prodromal, mild, moderate, or severe AD). In some embodiments, a patient has AD and has a confirmed amyloid pathology. In certain instances, the disclosure provides a method of treating a patient with prodromal Alzheimer's disease. In some instances, the disclosure provides a method of treating a patient with early Alzheimer's disease. In some instances, the disclosure provides a method of treating a patient to reduce clinical decline in Alzheimer's disease. In some instances, the disclosure provides a method of treating a patient with mild cognitive impairment due to Alzheimer's disease. In other instances, the disclosure provides a method of treating a patient with mild Alzheimer's disease dementia. In a further embodiment, the patient has amyloid pathology confirmed, e.g., by positron emission tomography (PET) imaging. In some cases, amyloid β pathology is confirmed by [¹⁸F]-florbetapir PET imaging. In some cases, amyloid β pathology is confirmed by [¹⁸F]-flutemetomol PET imaging. In some cases, amyloid β pathology is confirmed by [¹⁸F]-florbetaben PET imaging. In some cases, amyloid β pathology is confirmed by CSF amyloid β analysis. In some cases, amyloid β pathology is confirmed by blood amyloid β analysis. In some cases, amyloid β pathology is confirmed by Congo red staining and birefringence under polarized microscopy. In some cases, amyloid β pathology is confirmed by immunohistochemistry (IHC), electron microscopy, or mass spectrometry. In some cases, amyloid β pathology is confirmed by any method to assess levels of amyloid β.

In certain instances, the patient to be treated has an MMSE score between 24-30 (inclusive). In some instances, the patient to be treated has a CDR global score of 0.5. In some instances, the patient to be treated has a RBANS score of less than or equal to 85 (based upon Delayed Memory Index score). In some instances, the patient to be treated has at least 6 years of work experience. In some cases, the patient to be treated has an MMSE score between 24-30 (inclusive); a CDR global score of 0.5; and a RBANS score of less than or equal to 85 (based upon Delayed Memory Index score). In certain instances, the patient is an ApoE4 carrier (ApoE4 positive). In certain instances, the patient is an ApoE4 non-carrier (ApoE4 negative). In some embodiments, a patient is a human subject for whom the ApoE ε4 carrier status is known. In some embodiments, a patient is a human subject for whom the ApoE ε4 carrier status is not known.

In some embodiments, a patient is a human subject who has a confirmed amyloid beta pathology. In some embodiments, a baseline brain MRI has been obtained for the patient prior to performance of a method described herein. In some embodiments, a brain MRI was performed on the human subject within one year prior to performing a method described herein.

AD patients in need of treatment range from subjects with amyloid pathology and early neuronal degeneration to subjects with widespread neurodegeneration and irreversible neuronal loss with progressive cognitive and functional impairment to subjects with dementia.

Patients with preclinical AD can be identified by asymptomatic stages with or without memory complaints and emerging episodic memory and executive function deficits. This stage is typically characterized by the appearance of in vivo molecular biomarkers of AD and the absence clinical symptoms.

Prodromal AD patients are pre-dementia stage characterized predominantly by cognitive deficits and emerging functional impairment with disease progression. Prodromal AD patients typically have mini-mental state examination (MMSE) scores between 24-30 (inclusive), a spontaneous memory complaint, objective memory loss defined as a free recall score of <27 on the Free and Cued Selective Reminding Test (FCSRT), a global Clinical Dementia Rating (CDR) score of 0.5, absence of significant levels of impairment in other cognitive domains, and essentially preserved activities of daily living, and an absence of dementia.

Patients with mild AD typically have MMSE scores between 20-26 (inclusive), a global CDR of 0.5 or 1.0, and meet the National Institute on Aging-Alzheimer's Association core clinical criteria for probable AD (see Section 22).

Basing AD diagnosis on clinical symptoms, mild stage AD patients will exhibit conspicuous behavior at work, forgetfulness, mood swings, and attention disturbances. Moderate stage AD patients will exhibit cognitive deficits, restricted everyday activities, orientation disturbance, apraxia, agnosia, aphasia, and behavioral abnormalities. Severe stage AD patients are characterized by loss of independence, decay of memory and speech, and incontinence,

In certain embodiments, treatment is of earlier-stage patients who are amyloid positive as assessed by [¹⁸F]-florbetapir PET scans. In certain embodiments, treatment is of earlier-stage patients who are amyloid positive as assessed by ¹⁸F-flutemetomol PET scans. In certain embodiments, treatment is of earlier-stage patients who are amyloid positive as assessed by 18F-florbetaben PET scans. In certain instances, the human subject is confirmed to have a brain amyloid beta pathology prior to the initiation of treatment. The patient may be asymptomatic for, or exhibit only transient symptoms of, headache, confusion, gait difficulties, or visual disturbances. The patient may or may not be an ApoE4 carrier as determined by ApoE genotyping.

In other embodiments, treatment is of patients having any medical or neurological condition (other than AD) that might be a contributing cause of the subject's cognitive impairment, such as stroke or other cerebrovascular condition, other neurodegenerative disease, a history of clinically significant psychiatric illness, acute or sub-acute micro- or macro hemorrhage, prior macrohemorrhage, or superficial siderosis. These patients can be treated following screening and selection by a qualified clinician.

### Anti-Beta Amyloid Antibody

Antibody BIIB037, also known as aducanumab or ADUHELM, is a biologic treatment for Alzheimer's disease. It is an anti-Aβ antibody that recognizes aggregated forms of Aβ, including plaques. BIIB037 contains a human kappa light chain. BIIB037 consists of 2 heavy and 2 human kappa light chains connected by inter-chain disulfide bonds. By “BIIB037” or “aducanumab” or “ADUHELM” is meant an anti-Aβ antibody comprising the amino acid sequences set forth in SEQ ID NOs: 10 and 11.

The VH and VL of antibody BIIB037 have amino acid sequences that are identical to the amino acid sequence of the VH and VL of antibody NI-101.12F6A described in U.S. Pat. No. 8,906,367 (see, Tables 2-4; incorporated by reference in its entirety herein). Specifically, antibody BIIB037 has an antigen binding domain comprising VH and VL variable regions depicted in Table A (VH) and Table B (VL), corresponding complementarity determining regions (CDRs) depicted in Table C, and heavy and light chains depicted in Table D (H) and Table E (L).

TABLE A
Amino acid sequences of the V_{H} region of anti-Aβ antibody BIIB037
(VH CDRs (Kabat definition) underlined).
Variable heavy chain sequence
QVQLVESGGG VVQPGRSLRL SCAASGFAFS SYGMHWVRQA PGKGLEWVAV
IWFDGTKKYY TDSVKGRFTI SRDNSKNTLY LQMNTLRAED TAVYYCARDR
GIGARRGPYY MDVWGKGTTV TVSS (SEQ ID NO: 1)

TABLE B
Amino acid sequences of the V_{L} region of anti-Aβ antibody BIIB037
(VL CDRs (Kabat definition) underlined).
Variable light chain sequence (kappa or lambda)
DIQMTQSPSS LSASVGDRVT ITCRASQSIS SYLNWYQQKP GKAPKLLIYA
ASSLQSGVPS RFSGSGSGTD FTLTISSLQP EDFATYYCQQ SYSTPLTFGG
GTKVEIKR (SEQ ID NO: 2)

TABLE C
Denomination of CDR protein sequences in Kabat Nomenclature of
V_{H} and V_{L} regions of anti-Aβ antibody BIIB037.

| CDR | Variable heavy chain | Variable light chain |
|---|---|---|
| CDR1 | SYGMH (SEQ ID NO: 3) | RASQSISSYLN (SEQ ID NO: 6) |
| CDR2 | VIWFDGTKKYYTDSVKG (SEQ ID | AASSLQS (SEQ ID NO: 7) |
| | NO: 4) | |
| CDR3 | DRGIGARRGPYYMDV (SEQ ID NO: 5) | QQSYSTPLT (SEQ ID NO: 8) |

The amino acid sequence of the mature heavy chain of BIIB037 is provided in Table D below.

TABLE D
Amino acid sequences of the heavy chain of anti-Aβ antibody
BIIB037 (heavy chain CDRs (Kabat definition) underlined).
Heavy chain sequence
QVQLVESGGG VVQPGRSLRL SCAASGFAFS SYGMHWVRQA PGKGLEWVAV
IWFDGTKKYY TDSVKGRFTI SRDNSKNTLY LQMNTLRAED TAVYYCARDR
GIGARRGPYY MDVWGKGTTV TVSSASTKGP SVFPLAPSSK STSGGTAALG
CLVKDYFPEP VTVSWNSGAL TSGVHTFPAV LQSSGLYSLS SVVTVPSSSL
GTQTYICNVN HKPSNTKVDK RVEPKSCDKT HTCPPCPAPE LLGGPSVFLF
PPKPKDTLMI SRTPEVTCVV VDVSHEDPEV KFNWYVDGVE VHNAKTKPRE
EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT
TPPVLDSDGS FFLYSKLTVD KSRWQQGNVF SCSVMHEALH NHYTQKSLSL
SPG (SEQ ID NO: 10)

The amino acid sequence of the mature light chain of BIB037 is provided in Table E below.

TABLE E
Amino acid sequences of the light chain of anti-Aβ antibody BIIB037
(light chain CDRs (Kabat definition) underlined).
Light chain sequence
DIQMTQSPSS LSASVGDRVT ITCRASQSIS SYLNWYQQKP GKAPKLLIYA
ASSLQSGVPS RFSGSGSGTD FTLTISSLQP EDFATYYCQQ SYSTPLTFGG
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC (SEQ ID NO: 11)

In addition to antibody BIB037, this disclosure contemplates the use of the other anti-beta-amyloid antibodies, such as antibodies comprising either the VH region comprising or consisting of SEQ ID NO:1 or the VL region comprising or consisting of SEQ ID NO:2, or antibodies comprising the VH region comprising or consisting of SEQ ID NO:1 and the VL region comprising or consisting of SEQ ID NO:2, wherein the VH and/or VL regions have one or more substitutions, deletions, and/or insertions. In some embodiments, these VH and VL regions may have up to 25, up to 20, up to 15, up to 10, up to 5, or 1, 2, 3, 4, 5, 6, 7, 8, 9, 10, 11, 12, 13, 14, 15, 16, 17, 18, 19, or 20 amino acid substitutions and still bind beta-amyloid. In specific embodiments, these amino acid substitutions occur only in the framework region. In some embodiments, the amino acid substitution(s) is/are conservative amino acid substitutions. In certain embodiments, the VH and VL regions may include 1 to 5 (1, 2, 3, 4, 5) amino acid deletions and/or additions and still bind beta-amyloid. In certain embodiments, these deletions and/or additions are made at the N- and/or C-terminus of the VH and/or VL regions. In one embodiment, one amino acid is deleted and/or added at the N and/or C-terminus of the VH region. In one embodiment, one amino acid is deleted and/or added at the N and/or C-terminus of the VL region.

Other antibodies contemplated for use in the disclosure include antibodies comprising the variable heavy chain (VH) CDRs and the variable light chain (VL) CDRs in Table C. Thus, the anti-beta amyloid antibodies comprise the CDRs comprising or consisting of the amino acid sequences of SEQ ID NOs.: 3-8. In some embodiments, the anti-beta amyloid antibodies comprise the CDRs comprising or consisting of the amino acid sequences of SEQ ID NOs.: 4-8 and include as VH CDR 1 an amino acid sequence comprising or consisting of GFAFSSYGMH (SEQ ID NO:9). In some instances, the disclosure encompasses anti-beta-amyloid antibodies comprising the VH and VL CDRs of BIIB037 based on any CDR definition (e.g., Kabat, Chothia, enhanced Chothia, AbM, or contact definition). See, e.g., http://www.bioinf.org.uk/abs/index.html. In some embodiments, the disclosure encompasses anti-beta-amyloid antibodies comprising the VH and VL CDRs of BIIB037 based on the Chothia definition. In some embodiments, the disclosure encompasses anti-beta-amyloid antibodies comprising the VH and VL CDRs of BIIB037 based on the enhanced Chothia definition. In some embodiments, the disclosure encompasses anti-beta-amyloid antibodies comprising the VH and VL CDRs of BIIB037 based on the AbM definition. In some embodiments, the disclosure encompasses anti-beta-amyloid antibodies comprising the VH and VL CDRs of BIIB037 based on the contact definition.

Antibody BIIB037 and other anti-beta-amyloid antibodies employed in the methods of the disclosure can be prepared using known methods. In some embodiments, the antibody is expressed in a Chinese hamster ovary (CHO) cell line.

The maximum tolerated amount of an anti-Aβ antibody is that quantity of the antibody which will produce a clinically significant response in the treatment of Alzheimer's disease consistent with safety. The present disclosure recognizes that a safety concern in treating patients with an anti-beta-amyloid antibody is the occurrence of ARIA, including ARIA-E and/or ARIA-H. In some embodiments, provided methods make it possible to employ higher doses of an anti-Aβ antibody (e.g., BIIB037) for the treatment of patients for AD.

In some embodiments, an anti-Aβ antibody is administered to the patient by intravenous infusion following dilution into saline. In some embodiments, each intravenous infusion step in the titration regimen of the invention will typically take about 1 hour.

The dose ranges and other numerical values herein include a quantity that has the same effect as the numerically stated amount as indicated by treatment of Alzheimer's disease in the patient and a reduction in the incidence or susceptibility of the patient to ARIA when compared to an individual not treated by the method of the invention. At the very least, each numerical parameter should be construed in light of the number of significant digits, applying ordinary rounding techniques. In addition, any numerical value inherently contains certain errors from the standard deviation of its measurement and such values are within the scope of the invention.

### Compositions

Methods provided herein can include administration of a composition comprising an anti-beta-amyloid antibody as described herein. In some embodiments, such compositions comprise an anti-beta-amyloid antibody at a concentration of 50 mg/ml to 250 mg/ml; methionine at a concentration of 5 mM to 150 mM; Arg·HCl at a concentration of 50 mM to 200 mM; histidine at a concentration of 10 mM to 30 mM; PS80 at a concentration of 0.01% to 0.1%; and sucrose at a concentration of 0 to 3%. In some embodiments, a composition further comprises a thiol-containing antioxidant at a concentration of 0.02 mM to 4 mM. In some embodiments, a composition has a pH of 5.2 to 6.2. In some embodiments, a composition has a pH of 5.2 to 6.0. In some embodiments, a composition has a pH of 5.3 to 5.7. In some embodiments, a composition has a pH of 5.5. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

In some embodiments, a composition comprises an anti-beta-amyloid antibody at a concentration of 100 mg/ml; Arg·HCl at a concentration of 150 mM; methionine at a concentration of 10 mM; histidine at a concentration of 10 mM to 30 mM; PS80 at a concentration of 0.01% to 0.1%; and sucrose at a concentration of 0 to 3%.

In some embodiments, a composition comprises an anti-beta-amyloid antibody at a concentration of 100 mg/ml; Arg·HCl at a concentration of 150 mM; methionine at a concentration of 10 mM; histidine at a concentration of 20 mM; and PS80 at a concentration of 0.05%.

In some embodiments, a composition comprises an anti-beta-amyloid antibody at a concentration of 175 mg/ml; Arg·HCl at a concentration of 150 mM; methionine at a concentration of 10 mM; histidine at a concentration of 10 mM to 30 mM; PS80 at a concentration of 0.01% to 0.1%; and sucrose at a concentration of 0 to 3%. In some embodiments, a composition further comprises a thiol-containing antioxidant at a concentration of 0.02 mM to 4 mM. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

In some embodiments, a composition comprises an anti-beta-amyloid antibody at a concentration of 175 mg/ml; Arg·HCl at a concentration of 150 mM; methionine at a concentration of 10 mM; histidine at a concentration of 20 mM; and PS80 at a concentration of 0.05%. In some embodiments, a composition further comprises a thiol-containing antioxidant at a concentration of 0.02 mM to 4 mM. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

### Treatment

As used herein, the terms “treat” or “treatment” generally mean obtaining a desired pharmacological and/or physiological effect in the subject being administered the anti-beta amyloid antibody. Hence, the term “treatment” as used herein includes: (a) inhibiting AD, e.g. arresting its development; (b) relieving AD, e.g. causing regression of AD; or (c) prolonging survival as compared to expected survival if not receiving treatment.

In some embodiments, the treatment is therapeutic. In another embodiment, treatment has a disease modifying effect. This means that the treatment slows or delays the underling pathological or pathophysiological disease processes and there is an improvement in clinical signs and symptoms of AD relative to placebo.

In some embodiments, treatment results in symptomatic improvement. This may consist of enhanced cognition, more autonomy, and/or improvement in neuropsychiatric and behavioral dysfunction, even if for only a limited duration.

In some embodiments, the disclosure relates to methods for delaying clinical decline or progression of disease, or relief of symptoms. Delaying clinical decline or disease progression directly impacts the patient and care-givers. It delays disability, maintains independence, and allows the patient to live a normal life for a longer period of time. Relief of symptoms to the best degree possible can incrementally improve cognition, function, and behavioral symptoms, as well as mood.

This disclosure features a titration regimen (sequential administration of increasing doses of the anti-beta amyloid antibody) to treat Alzheimer's disease. One of the advantages of such a titration regimen is that it makes it possible to administer higher doses of the monoclonal antibody to AD patients without incurring the same extent of ARIA observed with a standard dose regimen. In some instances, the Alzheimer's disease is mild Alzheimer's disease, early Alzheimer's disease, prodromal Alzheimer's disease, mild Alzheimer's disease dementia, or mild cognitive impairment due to Alzheimer's disease.

In some methods of treatment of Alzheimer's disease of the present disclosure, the anti-beta amyloid antibody is administered to a human patient in increasing amounts over a period of time. This procedure of sequentially administering the antibody to the patient is referred to herein as “titration” because it involves administering a standardized pharmaceutical of known concentrations in carefully measured amounts until completion of the procedure.

In some instances, the disclosure provides methods for treating Alzheimer's disease in a human patient in need thereof, said methods comprising sequentially administering multiple doses of an anti-Aβ antibody (e.g., BIIB037) in increasing amounts over a period of time to the human patient, wherein multiple doses of 1 mg antibody/kg of body weight of the human patient are administered to the human patient at intervals of about 4 weeks; multiple doses of 3 mg antibody/kg of body weight of the human patient are administered to the human patient at intervals of about 4 weeks; multiple doses of 6 mg antibody/kg of body weight of the human patient are administered to the human patient at intervals of about 4 weeks; and multiple doses of 10 mg antibody/kg of body weight of the human patient are administered to the human patient at intervals of about 4 weeks. Multiple doses means at least two (e.g., 2, 3, 4, 5, 6, 7, 8, 9, 10, 11, 12, 123, 14, 15, 16, 17, 18, 19, 20, 21, 22, 23, 24, 25, 26, 27, 28, 29, 30 or more) doses.

In some embodiments, provided methods comprise sequentially administering multiple doses of an anti-Aβ antibody (e.g., BIIB037) in doses of 1 mg/kg, 3 mg/kg, 6 mg/kg, and 10 mg/kg, where doses are administered in approximately four week intervals. In some embodiments, doses 1 and 2 are at 1 mg/kg, doses 3 and 4 are at 3 mg/kg, doses 5 and 6 are at 6 mg/kg, and dose 7 and beyond are at 10 mg/kg.

One protocol according to the disclosure, designated Protocol A, comprises:
(A) administering the anti-beta amyloid antibody to the patient in an amount of 1 mg/kg of body weight of the patient;
(B) 4 weeks after step (A), administering the anti-beta amyloid antibody to the patient in an amount of 1 mg/kg of body weight of the patient;
(C) 4 weeks after step (B), administering the anti-beta amyloid antibody to the patient in an amount of 3 mg/kg of body weight of the patient;
(D) 4 weeks after step (C), administering the anti-beta amyloid antibody to the patient in an amount of 3 mg/kg of body weight of the patient;
(E) 4 weeks after step (D), administering the anti-beta amyloid antibody to the patient in an amount of 6 mg/kg of body weight of the patient;
(F) 4 weeks after step (E), administering the anti-beta amyloid antibody to the patient in an amount of 6 mg/kg of body weight of the patient; and
(G) in consecutive intervals of 4 weeks after step (F), administering the anti-beta amyloid antibody to the patient in an amount of 10 mg/kg of body weight of the patient.

In other words, Protocol A comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, antibody doses 3 and 4 are administered to the patient in an amount of 3 mg/kg of body weight. In four week intervals after administration of dose 4, doses 5 and 6 of the antibody are administered to the patient in an amount of 6 mg/kg of body weight. And then, four weeks after administration of dose 6, antibody dose 7 is administered to the patient in an amount of 10 mg/kg of body weight.

In some instances, after dose 7 of Protocol A, 5, 6, 7, 8, 9, or 10 doses of the anti-beta amyloid antibody in an amount of 10 mg/kg of body weight are administered to the patient. In certain instances, at least 10, at least 11, at least 12, at least 13, or at least 14 doses of the anti-beta amyloid antibody in an amount of 10 mg/kg of body weight of the subject are administered to the patient. In certain instances, at least 15, at least 16, at least 17, at least 18, at least 19, or at least 20 doses of the anti-beta amyloid antibody in an amount of 10 mg/kg of body weight of the subject are administered to the patient. In certain instances, 15 to 16, 15 to 17, 15 to 18, 15 to 19, 15 to 20, 15 to 21, 15 to 22, 15 to 23, 15 to 24, or 15 to 25 doses of the anti-beta amyloid antibody in an amount of 10 mg/kg of body weight of the subject are administered to the patient. In certain instances, the doses mentioned above are administered in consecutive intervals of 4 weeks. In certain instances, the doses mentioned above are administered to the patient intravenously.

In some instances, after dose 7 of Protocol A, at least 10 doses of the anti-beta amyloid antibody in an amount of 10 mg/kg of body weight of the subject are administered (e.g., intravenously) to the patient in uninterrupted 4 week intervals.

Another protocol according to the disclosure, designated Protocol B, comprises:
(a) administering the anti-beta-amyloid antibody to the subject in an amount of 1 mg/kg of body weight of the subject;
(b) 4 weeks after step (a), administering the anti-beta-amyloid antibody to the subject in an amount of 3 mg/kg of body weight of the subject;
(c) 4 weeks after step (b), administering the anti-beta-amyloid antibody to the subject in an amount of 6 mg/kg of body weight of the subject; and
(d) in consecutive intervals of 4 weeks after step (c), administering at least 10 doses of the anti-beta-amyloid antibody in an amount of 10 mg/kg of body weight of the subject.

In some instances, after step (d) of Protocol B, additional doses of the anti-beta amyloid antibody in an amount of 10 mg/kg of body weight are administered to the patient. In certain instances, at least 11, at least 12, at least 13, at least 14, at least 15, at least 16, at least 17, at least 18, at least 19, or at least 20 doses of the anti-beta amyloid antibody in an amount of 10 mg/kg of body weight of the subject are administered to the patient. In certain instances, at least 21, at least 22, at least 23, at least 24, at least 24, or at least 25 doses of the anti-beta amyloid antibody in an amount of 10 mg/kg of body weight of the subject are administered to the patient. In certain instances, 11 to 12, 11 to 13, 11 to 14, 11 to 15, 11 to 16, 11 to 17, 11 to 18, 11 to 19, 11 to 20, or 11 to 25 doses of the anti-beta amyloid antibody in an amount of 10 mg/kg of body weight of the subject are administered to the patient. In certain instances, the additional doses mentioned above are administered in consecutive intervals of 4 weeks. In certain instances, the doses mentioned above are administered to the patient intravenously.

In some instances, the anti-beta amyloid antibody of the protocols and methods above comprises a VH and VL comprising the six CDRs of BIIB037. In certain instances, the anti-beta amyloid antibody comprises the VH and VL of BIIB037. In other instances, the anti-beta amyloid antibody comprises the heavy and light chains of BIIB037. In some instances, the anti-beta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8. In some instances, the anti-beta amyloid antibody comprises a VH comprising or consisting of SEQ ID NO:1; and a VL comprising or consisting of SEQ ID NO:2. In some cases, the anti-beta-amyloid antibody comprises a heavy chain and a light chain, wherein: the heavy chain comprises or consists of SEQ ID NO:10; and the light chain comprises or consists of SEQ ID NO:11.

### Managing ARIA During Treatment with an Anti-Aβ Antibody

The present disclosure recognizes that methods of treatment of AD that include administering an anti-beta-amyloid antibody (e.g., BIIB037) can cause amyloid related imaging abnormalities-edema (ARIA-E) and -hemosiderin deposition (ARIA-H). The present disclosure further recognizes that occurrence of ARIA is a risk during the first 8 doses of treatment with anti-beta-amyloid antibody (e.g., BIIB037), particularly during titration.

This disclosure also provides methods of modifying the treatment of such patients. The methods can involve dose suspension, and/or dose modification, and/or termination of treatment with the anti-Aβ antibody.

In some embodiments, the disclosure provides methods for treating Alzheimer's disease that include monitoring for ARIA by obtaining brain magnetic resonance imaging (MRI) during treatment with the anti-beta-amyloid antibody.

The severity of clinical symptoms are defined as follows:

Mild: Symptom(s) barely noticeable to subject or does not make subject uncomfortable; does not influence performance or functioning; prescription drug not ordinarily needed for relief of symptom(s) but may be given because of personality of subject.

Moderate: Symptom(s) of a sufficient severity to make subject uncomfortable; performance of daily activity is influenced; subject is able to continue in study; treatment for symptom(s) may be needed.

Severe: Symptom(s) cause severe discomfort; symptoms cause incapacitation or significant impact on subject's daily life; severity may cause cessation of treatment with study treatment; treatment for symptom(s) may be given and/or subject hospitalized.

In some instances, seizure, including status epilepticus, has been associated with ARIA.

### Measurement of ARIA

AD patients generally respond to anti-Aβ antibody (e.g., BIIB037) in a dose dependent manner. Therefore, it is advantageous to use high doses for maximum effectiveness. The present disclosure encompasses a recognition that the incidence or rate of ARIA can increase when doses of the anti-Aβ antibody are increased, and particularly during the initial doses of anti-Aβ antibody. The present disclosure provides methods that mitigate the risk of ARIA to improve safety in treatment of patients with Alzheimer's disease. This disclosure makes it possible to manage the risk of amyloid related imaging abnormalities-edema (ARIA-E) and/or amyloid related imaging abnormalities-hemorrhage or hemosiderosis (ARIA-H).

ARIA, including edema (ARIA-E) and microhemorrhage or hemosiderosis (ARIA-H), are readily detectable by MRI (i.e., fluid attenuated inversion recovery (FLAIR/T2 for ARIA-E and T2*/gradient echo for ARIA-H). (Sperling R, et al. Amyloid-related imaging abnormalities in patients with Alzheimer's disease treated with bapineuzumab: a retrospective analysis. Lancet Neurol., 2012; 11(3):241-9). Susceptibility weighted imaging (SWI), an MRI technique potentially more sensitive than T2*/gradient echo in detecting ARIA-H (Sperling R A, et al. Amyloid-related imaging abnormalities in amyloid-modifying therapeutic trials: Recommendations from the Alzheimer's Association Research Roundtable Workgroup. Alzheimer's and Dementia, 2011; 7(4):367-85), can also be employed.

Signs of vasogenic edema include hyperintense signal on T2-weighted and FLAIR sequences generally confined to the white matter and often associated with gyral swelling. Symptoms of vasogenic edema when present include headache, worsening cognitive function, alteration of consciousness, seizures, unsteadiness, and vomiting.

ARIA-H is monitorable by MRI and believed to be an imaging finding without clinical correlate (i.e., patients are asymptomatic) (Sperling R A, et al. Amyloid-related imaging abnormalities in amyloid-modifying therapeutic trials: Recommendations from the Alzheimer's Association Research Roundtable Workgroup. Alzheimer's and Dementia, 2011; 7(4):367-85). Specifically, hemorrhage is detectable using MRI sequences of gradient echo, T1-weighted, T2-weighted, and FLAIR. Microhemorrhage is usually asymptomatic, whereas macrohemorrhage typically has focal signs and symptoms reflecting the area of the affected brain as well as non-specific symptoms that include those for vasogenic edema. The frequency of MRI acquisition is driven by safety monitoring needs.

In some embodiments, ARIA is monitored by MRI at particular time points. In some embodiments, an MRI is obtained and/or performed prior to the first dose of 6 mg/kg. In some embodiments, an MRI is obtained and/or performed prior to the first dose of 10 mg/kg. In some embodiments, an MRI is obtained and/or performed prior to the third dose of 10 mg/kg. In some embodiments, an MRI is obtained and/or performed prior to the sixth dose of 10 mg/kg.

In some embodiments, an MRI is obtained and/or performed prior to the first dose of 10 mg/kg and prior to the sixth dose of 10 mg/kg. In some embodiments, an MRI is obtained and/or performed at each of: (i) prior to the first dose of 6 mg/kg, (ii) prior to the first dose of 10 mg/kg, (iii) prior to the third dose of 10 mg/kg, and (iv) prior to the sixth dose of 10 mg/kg.

In some embodiments, provided methods include performing a brain MRI on the human subject prior to the fifth, seventh, ninth, and/or twelfth dose of the antibody. In some embodiments, provided methods include performing a brain MRI on the human subject prior to the seventh and twelfth doses of the antibody. In some embodiments, provided methods include performing a brain MRI on the human subject prior to each of the fifth, seventh, ninth, and twelfth doses of the antibody.

### Management of ARIA-E

In some embodiments, the present disclosure provides dosing recommendations for patients with ARIA-E as outlined in Table 6 of Example 4 below.

The severity of ARIA-E is defined as follows:

Mild ARIA-E: mild Fluid-attenuated inversion recovery (FLAIR) hyper-intensity confined to sulcus and/or cortex or subcortical white matter (with or without gyral swelling and sulcal effacement) which affects an area of less than 5 cm in single greatest dimension. Only a single region of involvement detected.

Moderate ARIA-E: moderate involvement area of FLAIR hyper-intensity measuring 5-10 cm in single greatest dimension, or more than one site of involvement, each measuring less than 10 cm in single greatest dimension.

Severe ARIA-E: severe involvement (area of FLAIR hyper-intensity measuring greater than 10 cm in single greatest dimension), often with significant subcortical white matter and/or sulcal involvement (with associated gyral swelling and sulcal effacement). One or more separate/independent sites of involvement may be noted.)

In some embodiments, the disclosure provides methods that include monitoring for ARIA-E by obtaining brain magnetic resonance imaging (MRI) during treatment with the anti-beta-amyloid antibody.

In some embodiments, where ARIA-E is detected in a human subject that is radiographically mild ARIA-E with mild clinical symptoms or no clinical symptoms, treatment is continued. In some embodiments, where the ARIA detected in the human subject is radiographically mild ARIA-E with clinical symptoms (e.g., moderate or severe clinical symptoms) or radiographically moderate or severe ARIA-E, and the treatment is suspended until radiographic resolution.

In some embodiments, a subject with ARIA-E also experiences seizure, such as, e.g., status epilepticus.

In some embodiments, the present disclosure provides methods that include (i) administering to a population of human subject multiple doses of an anti-beta-amyloid antibody; and (ii) suspending administration in those human subjects that develop radiographically moderate or severe ARIA-E during treatment. In some embodiments, the method further comprises resuming administration when a follow-up MRI demonstrates radiographic resolution.

In some embodiments, the present disclosure provides methods that include (i) administering to a population of human subject multiple doses of an anti-beta-amyloid antibody; and (ii) continuing administration in those human subjects that develop radiographically mild ARIA-E without symptoms or mild ARIA-E with mild symptoms during treatment.

### Management of ARIA-H

In some embodiments, the present disclosure provides dosing recommendations for patients with ARIA-H as outlined in Table 7 in Example 4 below.

ARIA-H includes microhemorrhage and superficial siderosis.

The severity of ARIA-H (microhemorrhage) is defined as follows:
Mild: 1-4 microhemorrhages
Moderate: 5-9 microhemorrhages
Severe: ≥10 microhemorrhages

The severity of ARIA-H (superficial siderosis) is defined as follows:
Mild Area of superficial siderosis: 1 new focal region
Moderate Area of superficial siderosis: 2 new focal regions
Severe Area of superficial siderosis: >2 new focal regions.

In some embodiments, the disclosure provides methods that include monitoring for ARIA-H by obtaining brain magnetic resonance imaging (MRI) during treatment with the anti-beta-amyloid antibody.

In some embodiments, the present disclosure provides methods that include (i) administering to a population of human subject multiple doses of an anti-beta-amyloid antibody; and (ii) suspending administration in those human subjects that develop radiographically mild ARIA-H with clinical symptoms or radiographically moderate or severe ARIA-H. In some embodiments, the method further comprises resuming administration when a follow-up MRI demonstrates radiographic stabilization.

In some embodiments, a subject with ARIA-H also experiences seizure, such as, e.g., status epilepticus.

In some embodiments, the present disclosure provides methods that include (i) administering to a population of human subject multiple doses of an anti-beta-amyloid antibody; and (ii) continuing administration in those human subjects that develop radiographically mild ARIA-H with no clinical symptoms during treatment.

In some embodiments, the present disclosure provides methods that include (i) administering multiple doses of an anti-beta-amyloid antibody to a population of human subjects with Alzheimer's disease, and (ii) in those subjects that develop ARIA-H (e.g., as detected by brain MRI) during treatment with the anti-beta-amyloid antibody: performing a follow-up brain MRI; and if the MRI demonstrates radiographic stabilization as indicated by no increase in size or number of ARIA-H, then administering further doses of the anti-beta-amyloid antibody to the human subjects, at a dose level not reduced compared to the last dose administered prior to the detection of ARIA-H. In some embodiments, the ARIA-H is radiographically mild ARIA-H with clinical symptoms or radiographically moderate or severe ARIA-H.

In some embodiments, the present disclosure provides methods that include: (i) administering multiple doses of an anti-beta-amyloid antibody to a population of human subjects with Alzheimer's disease, and (ii) in those subjects that develop intracerebral hemorrhage that is greater than 1 cm in diameter during treatment with anti-amyloid-beta antibody: permanently discontinuing administration of anti-beta-amyloid antibody.

The following are examples of the practice of the invention. They are not to be construed as limiting the scope of the invention in any way.

### EXAMPLES

### Example 1: Clinical Studies

The efficacy of ADUHELM was evaluated in two double-blind, randomized, placebo-controlled, parallel group studies (Study 1, NCT 02484547 and Study 2, NCT 02477800) in patients with Alzheimer's disease (patients with confirmed presence of amyloid pathology and mild cognitive impairment or mild dementia stage of disease, consistent with Stage 3 and Stage 4 Alzheimer's disease, stratified to include 80% Stage 3 patients and 20% Stage 4 patients). The effects of ADUHELM were also supported by a double-blind, randomized, placebo-controlled, doseranging study (Study 3, NCT 01677572) in patients with Alzheimer's disease (patients with confirmed presence of amyloid pathology and prodromal or mild dementia stage of disease, consistent with Stage 3 and Stage 4 Alzheimer's disease, with an enrolled distribution of 43% Stage 3 patients and 57% Stage 4 patients), followed by an optional, dose-blind, long-term extension period.

In Studies 1 and 2, patients were randomized to receive ADUHELM low dose (3 or 6 mg/kg for ApoE ε4 carriers and noncarriers, respectively), ADUHELM high dose (10 mg/kg), or placebo every 4 weeks for 18 months, followed by an optional, dose-blind, long-term extension period. Both studies included an initial titration period of up to 6 months to the maximum target dose. At the beginning of the study, ApoE ε4 carriers were initially titrated up to a maximum of 6 mg/kg in the high dose group, which was later adjusted to 10 mg/kg.

In Studies 1 and 2, patients were enrolled with a Clinical Dementia Rating (CDR) global score of 0.5, a Repeatable Battery for Assessment of Neuropsychological Status (RBANS) delayed memory index score ≤85, and a Mini-Mental State Examination (MMSE) score of 24-30. In Study 3, patients were enrolled with a global CDR score of 0.5 or 1.0 and an MMSE score of 20-30. Patients were enrolled with or without concomitant approved therapies (cholinesterase inhibitors and the N-methyl-D-aspartate antagonist memantine) for Alzheimer's disease.

### Example 2: Pharmacodynamics

### Effect of ADUHELM on Amyloid Beta Pathology

ADUHELM reduced amyloid beta plaque in a dose- and time-dependent manner in Study 1, Study 2, and Study 3, compared with placebo.

The effect of ADUHELM on amyloid beta plaque levels in the brain was evaluated using PET imaging (18F-florbetapir tracer). The PET signal was quantified using the Standard Uptake Value Ratio (SUVR) method to estimate brain levels of amyloid beta plaque in composites of brain areas expected to be widely affected by Alzheimer's disease pathology (frontal, parietal, lateral temporal, sensorimotor, and anterior and posterior cingulate cortices), compared to a brain region expected to be spared of such pathology (cerebellum). The SUVR was also expressed on the Centiloid scale.

In substudies of Study 1 and Study 2, ADUHELM reduced amyloid beta plaque levels in the brain, producing reductions at both ADUHELM low dose and high dose levels and at both Weeks 26 and 78 (p<0.0001), compared to placebo. The magnitude of reduction was time- and dose-dependent. In the long-term extension of Study 1 and Study 2, a continued decrease in brain amyloid beta plaque levels was observed at Week 132 in patients initially randomized to ADUHELM.

In Study 3, ADUHELM reduced amyloid beta plaque levels in the brain, producing statistically significant dose- and time-dependent reductions compared to placebo in the 3 mg/kg, 6 mg/kg, and 10 mg/kg ADUHELM treatment groups at Week 26, and in all ADUHELM treatment groups at Week 54. Among those dosed with ADUHELM during the placebo-controlled period in Study 3, amyloid beta plaque levels in the brain continued to decline in a time- and dosedependent manner in the long-term extension period through Week 222.

### Effect of ADUHELM on Tau Pathophysiology

ADUHELM reduced markers of tau pathophysiology (CSF p-Tau and Tau PET) and neurodegeneration (CSF t-Tau) in Study 1 and Study 2. ADUHELM reduced CSF levels of p-Tau in substudies conducted in Study 1 and Study 2. The adjusted mean change from baseline in CSF p-Tau levels relative to placebo was in favor of the ADUHELM low (p<0.01) and high (p<0.001) dose groups at Week 78 in Study 1. Results in Study 2 numerically favored ADUHELM but were not statistically significant.

ADUHELM reduced CSF levels of t-Tau in substudies conducted in Study 1 and Study 2. The adjusted mean change from baseline in CSF t-Tau levels relative to placebo was in favor of the ADUHELM low (p<0.05) and high (p<0.01) dose groups at Week 78 in Study 1. Results in Study 2 numerically favored ADUHELM but were not statistically significant.

Substudies were conducted in both Study 1 and Study 2 to evaluate the effect of ADUHELM on neurofibrillary tangles composed of tau protein using PET imaging (18F-MK6240 tracer). The PET signal was quantified using the SUVR method to estimate brain levels of tau in brain regions expected to be affected by Alzheimer's disease pathology (medial temporal, temporal, frontal, cingulate, parietal, and occipital cortices) in the study population compared to a brain region expected to be spared of such pathology (cerebellum). Data from the substudies were pooled, comprising 37 patients with longitudinal follow-up. The adjusted mean change from baseline in tau PET SUVR relative to placebo at follow-up was in favor of ADUHELM high dose in the medial temporal (p<0.001), temporal (p<0.05), and frontal (p<0.05) brain regions. No statistically significant differences were observed for the cingulate, parietal, or occipital cortices.

### Exposure-Response Relationships

Model based exposure-response analyses for Studies 1 and 2 demonstrated that higher exposures to ADUHELM were associated with greater reduction in clinical decline on CDR-SB, ADASCog13, and ADCS-ADL-MCI. In addition, higher exposures to ADUHELM were associated with greater reduction in amyloid beta plaque in Studies 1 and 2. An association between reduction in amyloid beta plaque and clinical decline on CDR-SB was also observed.

### Example 3: Adverse Reactions

### Clinical Trials Experience

Because clinical trials are conducted under widely varying conditions, adverse reaction rates observed in clinical trials of a drug cannot be directly compared to rates in the clinical trials of another drug and may not reflect the rates observed in clinical practice.

The safety of ADUHELM has been evaluated in 3,078 patients who received at least one dose of ADUHELM. In two placebo-controlled studies (Studies 1 and 2) in patients with Alzheimer's disease, a total of 1105 patients received ADUHELM 10 mg/kg. Of these 1105 patients, approximately 52% were female, 76% were White, 10% were Asian, and 3% were of Hispanic or Latino ethnicity. The mean age at study entry was 70 years (range from 50 to 85).

In the combined placebo-controlled and long-term extension periods of Studies 1 and 2,834 patients received at least one dose of ADUHELM 10 mg/kg once monthly for at least 6 months, 551 patients for at least 12 months, and 309 patients for at least 18 months. In the combined placebo-controlled and long-term extension periods, 5% (66 out of 1386) of patients in the 10 mg/kg dose group withdrew from the study because of an adverse reaction. The most common adverse reaction resulting in study withdrawal in the combined placebo-controlled and long-term extension periods was ARIA-H superficial siderosis. Table 1 shows adverse reactions that were reported in at least 2% of patients treated with ADUHELM and at least 2% more frequently than in patients on placebo.

TABLE 1
Adverse Reactions Reported in at Least 2% of Patients
Treated with ADUHELM 10 mg/kg and at Least 2%
Higher Than Placebo in Studies 1 and 2

| | ADUHELM | |
|---|---|---|
| | 10 mg/kg | Placebo |
| | N = 1105 | N = 1087 |
| Adverse Reaction | % | % |
| ARIA-E | 35 | 3 |
| Headache^{a} | 21 | 16 |
| ARIA-H microhemorrhage | 19 | 7 |
| ARIA-H superficial siderosis | 15 | 2 |
| Fall | 15 | 12 |
| Diarrhea^{b} | 9 | 7 |
| Confusion/Delirium/Altered Mental | 8 | 4 |
| Status/Disorientation^{c} | | |

^{a}Headache includes the adverse reaction related terms headache, head discomfort, migraine, migraine with aura, and occipital neuralgia.
^{b}Diarrhea includes the adverse reaction related terms diarrhea and infectious diarrhea.
^{c}Confusion/Delirium/Altered Mental Status/Disorientation includes the adverse reaction related terms confusional state, delirium, altered state of consciousness, disorientation, depressed level of consciousness, disturbance in attention, mental impairment, mental status changes, postoperative confusion, and somnolence.

### Amyloid Related Imaging Abnormalities

ADUHELM can cause amyloid related imaging abnormalities-edema (ARIA-E), which can be observed on MRI as brain edema or sulcal effusions, and amyloid related imaging abnormalities hemosiderin deposition (ARIA-H), which includes microhemorrhage and superficial siderosis.

In clinical studies of ADUHELM, the severity of ARIA was classified by radiographic criteria, as shown in Table 2.

TABLE 2
ARIA MRI Classification Criteria

| | Radiographic Severity | | |
|---|---|---|---|
| ARIA Type | Mild | Moderate | Severe |
| ARIA-E | FLAIR hyperintensity | FLAIR | FLAIR hyperintensity |
| | confined to sulcus and | hyperintensity 5 to | measuring > 10 cm, often |
| | or cortex/subcortical | 10 cm, or more than | with significant subcortical |
| | white matter in one | 1 site of | white matter and/or sulcal |
| | location < 5 cm | involvement, each | involvement. One or more |
| | | measuring < 10 cm | separate sites of |
| | | | involvement may be noted. |
| ARIA-H | ≤4 new incident | 5 to 9 new incident | 10 or more new incident |
| microhemorrhage | microhemorrhages | microhemorrhages | microhemorrhages |
| ARIA-H | 1 focal area of | 2 focal areas of | >2 focal areas of |
| superficial | superficial siderosis | superficial siderosis | superficial siderosis |
| siderosis | | | |

In Studies 1 and 2, ARIA(-E and/or -H) was observed in 41% of patients treated with ADUHELM with a planned dose of 10 mg/kg (454 out of 1105), compared to 10% of patients on placebo (111 out of 1087).

ARIA-E was observed in 35% of patients treated with ADUHELM 10 mg/kg, compared to 3% of patients on placebo.

Studies 1 and 2, 16% (181/1105) of patients in the ADUHELM 10 mg/kg group were apolipoprotein E ε4 (ApoE ε4) homozygotes, 51% (568/1105) were heterozygotes, and 32% (356/1105) were noncarriers. In these studies, randomization was stratified by ApoE ε4 carrier status (i.e., carrier or non-carrier); therefore, interpretation of analyses by ApoE ε4 homozygous and heterozygous carrier status should consider the limitations of the unbalanced subgroups and the small number of homozygotes enrolled in the study. The incidence of ARIA-E was higher in ApoE ε4 carriers than in ApoE ε4 non-carriers (64% in homozygotes, 35% in heterozygotes, and 20% in noncarriers). Severe radiographic ARIA-E occurred in 11% of homozygotes, 4% of heterozygotes, and 2% of noncarriers. However, the incidence of serious adverse reactions with ARIA-E, including risk of death, persistent or significant disability or incapacity, hospitalization, or other medically important event that may require intervention to prevent serious outcomes, was similar for ApoE ε4 carriers and non-carriers (2% in homozygotes, 1% in heterozygotes, 2% in non-carriers). Testing for ApoE ε4 carrier status may be considered when initiating treatment with ADUHELM to inform the risk of developing ARIA. The recommendations on management of ARIA do not differ between ApoE ε4 carriers and non-carriers.

The majority of ARIA-E radiographic events occurred early in treatment (within the first 8 doses), although ARIA can occur at any time. Among patients treated with a planned dose of ADUHELM 10 mg/kg who had ARIA-E, the maximum radiographic severity was mild in 30%, moderate in 58%, and severe in 13% of patients. Resolution occurred in 68% of ARIA-E patients by 12 weeks, 91% by 20 weeks, and 98% overall after detection. 10% of all patients who received ADUHELM 10 mg/kg had more than one episode of ARIA-E.

ARIA-H in the setting of ARIA-E associated with the use of ADUHELM 10 mg/kg was observed in 21% of patients treated with ADUHELM 10 mg/kg, compared to 1% of patients on placebo. There was no imbalance in isolated ARIA-H (i.e., ARIA-H in patients who did not also experience ARIA-E) between ADUHELM and placebo. There was no imbalance in hemorrhage greater than 1 cm between ADUHELM and placebo. Intracerebral hemorrhage (greater than 1 cm in diameter) was reported in 0.5% of patients after treatment with ADUHELM 10 mg/kg compared to 0.4% of patients on placebo.

Patients were excluded from enrollment in Studies 1 and 2 for the following criteria: prior intracerebral hemorrhage greater than 1 cm in diameter, more than 4 microhemorrhages, superficial siderosis, history of diffuse white matter disease, and use of antiplatelet or anticoagulant medications other than 325 mg or less daily of aspirin. Although patients were allowed to receive aspirin in daily doses of 325 mg or less, some patients, because of intercurrent medical events that occurred after enrollment and required treatment, received aspirin in doses greater than 325 mg, other antiplatelet drugs or anticoagulants during Studies 1 and 2. In patients treated with 10 mg/kg ADUHELM during the placebo-controlled portion of the studies, those who received any antithrombotic medication (aspirin at any dose, other antiplatelet drugs, or anticoagulants; n=4305) did not have an increased risk for ARIA or intracerebral hemorrhage when compared with those who did not receive any antithrombotic medication (n=66570). The majority of exposures to antithrombotic medications were to aspirin; 77 patients were exposed to other antiplatelet drugs or anticoagulants, limiting any definitive conclusions about the risk of ARIA or intracerebral hemorrhage in these patients.

Clinical symptoms were present in 24% of patients treated with ADUHELM 10 mg/kg who had an observation of ARIA(-E and/or -H), compared to 5% of patients on placebo. The most common symptom in patients treated with ADUHELM 10 mg/kg with ARIA was headache (13%). Other frequent symptoms were confusion/delirium/altered mental status/disorientation (5%), dizziness/vertigo (4%), visual disturbance (2%), and nausea (2%). Serious symptoms associated with ARIA were reported in 0.3% of patients treated with ADUHELM 10 mg/kg. Clinical symptoms resolved in the majority of patients (88%) during the period of observation.

Seizure, including status epilepticus, which can be serious and life-threatening, has been associated with ARIA. The overall incidence of seizure, independent of ARIA, was 0.5% in the 10 mg/kg ADUHELM group and 0.8% in the placebo group in Studies 1 and 2. In patients with ARIA in the 10 mg/kg ADUHELM group, the incidence of seizure was 0.7%. Status epilepticus was reported in the placebo-controlled and long-term extension studies in patients treated with ADUHELM. In patients with status epilepticus, radiographically severe ARIA was observed preceding or shortly after the event.

Table 3 shows the cumulative proportions of first ARIA-E events, symptomatic first ARIA-E events, and first serious ARIA-E events in the high dose group (10 mg/kg) of Study 1 and Study 2 by number of infusions of ADUHELM. The cumulative proportion of first ARIA-E events reached 51% after 6 infusions (prior to the 7th dose) and 90% after 11 infusions (prior to the 12th dose). The cumulative proportion of first symptomatic ARIA-E events was similar, reaching 54% and 92% at the same two time points, while all events of first serious ARIA-E were observed prior to the 12th dose. These data support the benefit of monitoring for ARIA-E by MRI prior to the first dose of 10 mg/kg and the sixth dose of 10 mg/kg.

TABLE 3
First ARIA-E by Infusion Number

| | BIIB037 10 mg/kg (N = 1105) | | | | | |
|---|---|---|---|---|---|---|
| | First ARIA-E | | First symptomatic ARIA-E | | First serious ARIA-E | |
| Infusion | Total number of events | | | | | |
| number | 387 | | 101 | | 15 | |
| 1 | 0 | [0] | 0 | [0] | 0 | [0] |
| 2 | 1 (<1) | [1 (<1)] | 1 (<1) | [1 (<1)] | 1 (7) | [1 (7)] |
| 3 | 4 (1) | [5 (1)] | 2 (2) | [3 (3)] | 2 (13) | [3 (20)] |
| 4 | 88 (23) | [93 (24)] | 28 (28) | [31 (31)] | 6 (40) | [9 (60)] |
| 5 | 11 (3) | [104 27)] | 4 (4) | [35 (35)] | 2 (13) | [11 (73)] |
| 6 | 94 (24) | [198 (51)] | 20 (20) | [55 (54)] | 2 (13) | [13 (87)] |
| 7 | 5 (1) | [203 (52)] | 1 (<1) | [56 (55)] | 0 | [13 (87)] |
| 8 | 81 (21) | [284 (73)] | 17 (17) | [73 (72)] | 0 | [13 (87)] |
| 9 | 1 (<1) | [285 (74)] | 0 | [73 (72)] | 0 | [13 (87)] |
| 10 | 8 (2) | [293 (76)] | 5 (5) | [78 (77)] | 1 (7) | [14 (93)] |
| 11 | 54 (14) | [347 (90)] | 15 (15) | [93 (92)] | 1 (7) | [15 (100)] |
| 12 | 3 (<1) | [350 (90)] | 3 (3) | [96 (95)] | 0 | [15 (100)] |
| 13 | 2 (<1) | [352 (91)] | 1 (<1) | [97 (96)] | 0 | [15 (100)] |
| 14 | 16 (4) | [368 (95)] | 4 (4) | [101 (100)] | 0 | [15 (100)] |
| 15 | 0 | [368 (95)] | 0 | [101 (100)] | 0 | [15 (100)] |
| 16 | 1 (<1) | [369 (95)] | 0 | [101 (100)] | 0 | [15 (100)] |
| 17 | 8 (2) | [377 (97)] | 0 | [101 (100)] | 0 | [15 (100)] |
| 18 | 0 | [377 (97)] | 0 | [101 (100)] | 0 | [15 (100)] |
| 19 | 1 (<1) | [378 (98)] | 0 | [101 (100)] | 0 | [15 (100)] |
| 20 | 9 (2) | [387 (100)] | 0 | [101 (100)] | 0 | [15 (100)] |

NOTE 1:
Numbers in parentheses are percentages based on the number of events. Numbers in brackets present [cumulative frequency (cumulative percent)]. Event is counted in the nth row if after the nth dose but before the (n + 1)th dose.
NOTE 2:
ApoE e4 carriers in the SCS 6 mg/kg group analyzed in the 10 mg/kg group.

Table 4 shows the cumulative proportions of first ARIA of any type (-E and/or -H) in the high dose group (10 mg/kg) of Study 1 and Study 2 by number of infusions of ADUHELM. Similar to what was seen for first ARIA-E, 50% of first ARIA was observed prior to the 7th dose and 86% prior to the 12th dose. For first symptomatic ARIA events, 56% and 91% occurred prior the 7th and 12th doses, respectively, while all first serious ARIA events occurred prior to the 12th dose. These data support the benefit of monitoring for ARIA (both ARIA-E and ARIA-H) by MRI prior to the first dose of 10 mg/kg and the sixth dose of 10 mg/kg.

TABLE 4
First ARIA by Infusion Number

| | BIIB037 10 mg/kg (N = 1105) | | | | | |
|---|---|---|---|---|---|---|
| | First ARIA | | First symptomatic ARIA | | First serious ARIA | |
| Infusion | Total number of events | | | | | |
| number | 454 | | 110 | | 16 | |
| 1 | 0 | [0] | 0 | [0] | 0 | [0] |
| 2 | 1 (<1) | [1 (<1)] | 1 (<1) | [1 (<1)] | 1 (6) | [1 (6)] |
| 3 | 7 (2) | [8 (2)] | 2 (2) | [3 (3)] | 2 (13) | [3 (19)] |
| 4 | 106 (23) | [114 (25)] | 34 (31) | [37 (34)] | 6 (38) | [9 (56)] |
| 5 | 11 (2) | [125 (28)] | 4 (4) | [41 (37)] | 2 (13) | [11 (69)] |
| 6 | 101 (22) | [226 (50)] | 20 (18) | [61 (55)] | 2 (13) | [13 (81)] |
| 7 | 7 (2) | [233 (51)] | 1 (<1) | [62 (56)] | 0 | [13 (81)] |
| 8 | 91 (20) | [324 (71)] | 18 (16) | [80 (73)] | 1 (6) | [14 (88)] |
| 9 | 0 | [324 (71)] | 0 | [80 (73)] | 0 | [14 (88)] |
| 10 | 11 (2) | [335 (74)] | 5 (5) | [85 (77)] | 1 (6) | [15 (94)] |
| 11 | 55 (12) | [390 (86)] | 15 (14) | [100 (91)] | 1 (6) | [16 (100)] |
| 12 | 5 (1) | [395 (87)] | 3 (3) | [103 (94)] | 0 | [16 (100)] |
| 13 | 3 (<1) | [398 (88)] | 1 (<1) | [104 (95)] | 0 | [16 (100)] |
| 14 | 27 (6) | [425 (94)] | 5 (5) | [109 (99)] | 0 | [16 (100)] |
| 15 | 0 | [425 (94)] | 0 | [109 (99)] | 0 | [16 (100)] |
| 16 | 2 (<1) | [427 (94)] | 0 | [109 (99)] | 0 | [16 (100)] |
| 17 | 12 (3) | [439 (97)] | 1 (<1) | [110 (100)] | 0 | [16 (100)] |
| 18 | 2 (<1) | [441 (97)] | 0 | [110 (100)] | 0 | [16 (100)] |
| 19 | 1 (<1) | [442 (97)] | 0 | [110 (100)] | 0 | [16 (100)] |
| 20 | 12 (3) | [454 (100)] | 0 | [110 (100)] | 0 | [16 (100)] |

NOTE 1:
Numbers in parentheses are percentages based on the total number of events. Numbers in brackets present [cumulative frequency (cumulative percent)]. Event is counted in the nth row if after the nth dose but before the (n + 1)th dose.
NOTE 2:
ApoE e4 carriers in the SCS 6 mg/kg group analyzed in the 10 mg/kg group.

Although the protocols for Study 1 and Study 2 specified dose suspension for certain radiographic ARIA findings, and therefore there are no systematic data on continued dosing with ADUHELM following detection of radiographically moderate or severe ARIA (or symptomatic ARIA), data generated during the studies regarding clinical outcomes in patients who were dosed with mild asymptomatic ARIA, or with moderate or severe radiographic ARIA, indicated that that dose suspension may not reduce the risk of serious clinical outcomes. Furthermore, it is recognized that dose suspension may delay the time to achieving sustained decline in amyloid beta and the accompanying clinical response. Therefore, considering the benefit risk of dosing with ADUHELM, and the data in Tables 3 and 4, a combination of symptom-triggered MRIs and two routine MRIs, conducted prior to the 7th and 12th doses, enables the identification of both symptomatic and asymptomatic ARIA events.

Enhanced clinical vigilance for ARIA is recommended during the first 8 doses of treatment with ADUHELM, particularly during titration, as this is the time the majority of ARIA was observed in Studies 1 and 2. If a patient experiences symptoms which could be suggestive of ARIA, clinical evaluation should be performed, including MRI testing if indicated. If ARIA is observed on MRI in the presence of clinical symptoms, careful clinical evaluation should be performed prior to continuing treatment.

Obtain brain MRIs prior to the 7th infusion (first dose of 10 mg/kg) and 12th infusion (sixth dose of 10 mg/kg) of ADUHELM to evaluate for the presence of asymptomatic ARIA. For patients with radiographic findings of ARIA, enhanced clinical vigilance is recommended. Additional MRIs may be considered if clinically indicated. If radiographically severe ARIA-H is observed, treatment may be continued with caution only after a clinical evaluation and a follow-up MRI demonstrates radiographic stabilization (i.e., no increase in size or number of ARIA-H). For ARIA-E or mild/moderate ARIA-H, treatment may continue with caution. If dosing is temporarily suspended, dosing may resume at that same dose and titration schedule. There are no systematic data on continued dosing with ADUHELM following detection of radiographically moderate or severe ARIA. In Studies 1 and 2, temporary dose suspension was required for radiographically moderate or severe ARIA-E and radiographically moderate ARIA-H. In Studies 1 and 2, permanent discontinuation of dosing was required for radiographically severe ARIA-H. The benefits of reaching and maintaining the 10 mg/kg dose should be considered when evaluating a potential dose suspension.

### Hypersensitivity Reactions

Angioedema and urticaria were reported in one patient in the placebo-controlled period of Studies 1 and 2, and occurred during the ADUHELM infusion.

### Example 4: Dosing Instructions

After an initial titration, the recommended dosage of ADUHELM is 10 mg/kg (see Table 5). ADUHELM is administered as an intravenous (IV) infusion over approximately one hour every four weeks and at least 21 days apart.

In one embodiment, ADUHELM is administered as an intravenous infusion over approximately one hour. In another embodiment, ADUHELM is administered via a 0.2 or 0.22 micron in-line filter.

TABLE 5
Dosing Schedule

| | ADUHELM Dosage | |
|---|---|---|
| IV Infusion | (administered over | |
| (every 4 weeks) | approximately one hour) | |
| Infusion 1 and 2 | 1 | mg/kg |
| Infusion 3 and 4 | 3 | mg/kg |
| Infusion 5 and 6 | 6 | mg/kg |
| Infusion 7 and beyond | 10 | mg/kg |

### Monitoring for Amyloid Related Imaging Abnormalities

Obtain recent (within one year) brain magnetic resonance imaging (MRI) prior to initiating treatment. Obtain MRIs prior to the 7th infusion (first dose of 10 mg/kg) and 12th infusion (sixth dose of 10 mg/kg). If 10 or more new incident microhemorrhages or >2 focal areas of superficial siderosis (radiographic severe ARIA-H) is observed, treatment may be continued with caution only after a clinical evaluation and a follow-up MRI demonstrates radiographic stabilization (i.e., no increase in size or number of ARIA-H).

### Resuming ADUHELM After Missed Dose

If an infusion is missed, resume administration at the same dose as soon as possible. Infusions are to be administered every 4 weeks and at least 21 days apart.

### Hypersensitivity Reactions

Angioedema and urticaria were reported in one patient. Promptly discontinue the infusion upon the first observation of any signs or symptoms consistent with a hypersensitivity reaction, and initiate appropriate therapy.

### Recommendations for Dosing Interruptions in Patients with ARIA

If dosing is resumed following a temporary suspension, dosing may resume at that same dose and titration schedule prior to the dosing suspension. The benefits of reaching and maintaining the 10 mg/kg dosage should be considered when evaluating a potential dose suspension.

The dosing interruptions for patients with ARIA-E are provided in Table 6.

TABLE 6
Dosing Recommendations for Patients with ARIA-E

| Clinical Symptom | ARIA-E Severity on MRI | | |
|---|---|---|---|
| Severity | Mild | Moderate | Severe |
| Asymptomatic | May continue dosing at | Suspend | Suspend |
| | current dose and schedule | dosing¹ | dosing¹ |
| Mild | May continue based | Suspend | |
| | on clinical evaluation | dosing¹ | |

| | |
|---|---|
| Moderate or Severe | Suspend dosing¹ |

¹Suspend until symptoms resolve and MRI demonstrates radiographic resolution

### ARIA-H

The dosing interruptions for patients with ARIA-H are provided in Table 7.

TABLE 7
Dosing Recommendations for Patients with ARIA-H

| Clinical Symptom | ARIA-H Severity on MRI | | |
|---|---|---|---|
| Severity | Mild | Moderate | Severe |
| Asymptomatic | May continue based | Suspend dosing¹ | Suspend |
| | on clinical evaluation | | dosing² |
| Symptomatic | Suspend dosing¹ | Suspend dosing¹ | |

¹Suspend until symptoms resolve and MRI demonstrates radiographic resolution.
²Suspend until symptoms resolve and MRI demonstrates radiographic stabilization.

Patients who develop brain hemorrhage that is greater than 1 cm in diameter during treatment with anti-amyloid-beta antibody (e.g., ADUHELM) should permanently discontinue treatment. Alternatively, in patients who develop intracerebral hemorrhage greater than 1 cm in diameter during treatment with anti-amyloid-beta antibody (e.g., ADUHELM), suspend dosing until MRI demonstrates radiographic stabilization and symptoms, if present, resolve.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating Alzheimer's disease in a human subject in need thereof comprising:
administering to the human subject multiple doses of an anti-beta-amyloid antibody,
monitoring for ARIA by obtaining brain magnetic resonance imaging (MRI) during treatment with the anti-beta-amyloid antibody, and
if ARIA is detected in the human subject:
continuing administration of the antibody if the ARIA detected is (i) radiographically mild ARIA-E with mild clinical symptoms or no clinical symptoms or (ii) radiographically mild ARIA-H with no clinical symptoms, or
suspending administration of the antibody if the ARIA detected is (i) radiographically moderate or severe ARIA-E, (ii) radiographically mild ARIA-H with clinical symptoms, or (iii) radiographically moderate or severe ARIA-H.

2. The method of claim 1, wherein the monitoring for ARIA comprises obtaining brain MRI prior to the first dose of 6 mg/kg, prior to the first dose of 10 mg/kg, prior to the third dose of 10 mg/kg, and/or prior to the sixth dose of 10 mg/kg.

3. The method of claim 1 or 2, wherein the administration is suspended and the method further comprises performing a follow-up brain magnetic resonance imaging (MRI).

4. The method of claim 3, wherein the follow-up brain MRI demonstrates radiographic stabilization and/or radiographic resolution and the administering is resumed at the same dose or a higher dose than was administered prior to the detection of ARIA.

5. A method for treating Alzheimer's disease in a human subject in need thereof, the method comprising:
administering to the human subject multiple doses of an anti-beta-amyloid antibody, wherein the human subject develops Amyloid Related Imaging Abnormality-H (ARIA-H) as detected by brain magnetic resonance imaging (MRI) during treatment with the anti-beta-amyloid antibody, wherein the ARIA-H comprises ten or more new incident microhemorrhages or greater than two focal areas of superficial siderosis, and
administering further doses of the anti-beta-amyloid antibody to the human subject, at a dose level not reduced compared to the last dose administered prior to the detection of ARIA-H, only after a clinical evaluation and a follow-up brain MRI demonstrates radiographic stabilization as indicated by no increase in size or number of ARIA-H.

6. The method of any one of claims 1-5, wherein the multiple doses of the anti-beta-amyloid antibody are administered intravenously to the human subject.

7. The method of any one of claims 1-6, wherein the multiple doses of the anti-beta-amyloid antibody are administered to the human subject at intervals of once every four weeks.

8. The method of any one of claims 1-7, wherein the multiple doses of the anti-beta-amyloid antibody comprise administering at least two doses of the antibody in an amount of 1 mg/kg of body weight of the human subject.

9. The method of any one of claims 1-8, wherein the multiple doses of the anti-beta-amyloid antibody comprise administering at least two doses of the antibody in an amount of 3 mg/kg of body weight of the human subject.

10. The method of any one of claims 1-9, wherein the multiple doses of the anti-beta-amyloid antibody comprise administering at least two doses of the antibody in an amount of 6 mg/kg of body weight of the human subject.

11. The method of any one of claims 1-10, wherein the multiple doses of the anti-beta-amyloid antibody comprise administering at least two doses of the antibody in an amount of 10 mg/kg of body weight of the human subject.

12. The method of claim 11, wherein the monitoring for ARIA comprises obtaining brain MRI prior to the first dose of 6 mg/kg, prior to the first dose of 10 mg/kg, prior to the third dose of 10 mg/kg, and/or prior to the sixth dose of 10 mg/kg.

13. The method of claim 11, wherein the monitoring for ARIA comprises obtaining brain MRI prior to the first dose of 10 mg/kg and prior to the sixth dose of 10 mg/kg.

14. The method of claim 11, wherein the monitoring for ARIA comprises obtaining brain MRI prior to each of: the first dose of 6 mg/kg, the first dose of 10 mg/kg, the third dose of 10 mg/kg, and the sixth dose of 10 mg/kg.

15. The method of any one of claims 1-14, wherein the multiple doses of the anti-beta-amyloid antibody comprise in four week intervals:
doses 1 and 2 of the antibody in an amount of 1 mg antibody/kg of body weight of the human subject;
doses 3 and 4 of the antibody in an amount of 3 mg antibody/kg of body weight of the human subject;
doses 5 and 6 of the antibody in an amount of 6 mg antibody/kg of body weight of the human subject;
doses 7, 8, 9, 10, 11, and 12 of the antibody in an amount of 10 mg antibody/kg of body weight of the human subject; and
maintenance doses of the antibody in an amount of 10 mg antibody/kg of body weight of the human subject.

16. A method for treating Alzheimer's disease in a human subject in need thereof, comprising:
administering a first dose of an anti-beta-amyloid antibody to the human subject in an amount of 1 mg antibody/kg of body weight of the human subject, followed by a second dose in an amount of 1 mg antibody/kg of body weight of the human subject four weeks after the first dose;
in four week intervals after the second dose, administering doses 3 and 4 to the human subject in an amount of 3 mg antibody/kg of body weight of the human subject;
in four week intervals after administration of dose 4, administering doses 5 and 6 of the antibody to the human subject in an amount of 6 mg antibody/kg of body weight of the human subject;
in four week intervals after administration of dose 6, administering doses 7, 8, 9, 10, 11, and 12 of the antibody to the human subject in an amount of 10 mg antibody/kg of body weight of the human subject; and
in four week intervals after administration of dose 12, administering maintenance doses of the antibody to the human subject in an amount of 10 mg antibody/kg of body weight of the human subject,
wherein a brain MRI is performed on the human subject within one year prior to administering dose 1 of the antibody,
wherein a brain MRI is performed on the human subject after administering dose 6 of the antibody and prior to administering dose 7 of the antibody, and
wherein a brain MRI is performed on the human subject after administering dose 11 of the antibody and prior to administering dose 12 of the antibody.

17. The method of claim 16, wherein the human subject develops ARIA-H as detected by brain MRI during treatment with the anti-beta-amyloid antibody, wherein the ARIA-H comprises ten or more new incident microhemorrhages or greater than two focal areas of superficial siderosis, and wherein the method comprises administering further doses of the anti-beta-amyloid antibody to the human subject, at a dose level not reduced compared to the last dose administered prior to the detection of ARIA-H, only after a clinical evaluation and a follow-up brain MRI demonstrates radiographic stabilization as indicated by no increase in size or number of ARIA-H.

18. The method of claim 17, wherein the ARIA-H is detected by the brain MRI performed on the human subject after prior to administering dose 5 of the antibody, dose 7 of the antibody, dose 9 of the antibody, and/or dose 12 of the antibody.

19. The method of claim 17, wherein the ARIA-H is detected by the brain MRI performed on the human subject after administering dose 6 of the antibody and prior to administering dose 7 of the antibody.

20. The method of claim 17, wherein the ARIA-H is detected by the brain MRI performed on the human subject after administering dose 11 of the antibody and prior to administering dose 12 of the antibody.

21. A method for treating Alzheimer's disease in a human subject in need thereof, the method comprising:
administering to the human subject multiple doses of an anti-beta-amyloid antibody; and
monitoring the human subject for ARIA during the first 8 doses of an anti-beta-amyloid antibody.

22. The method of claim 21, wherein the ARIA is ARIA-H.

23. The method of claim 22, wherein ARIA-H is detected by a brain MRI performed on the human subject after administering dose 4 of the antibody and prior to administering dose 5 of the antibody.

24. The method of claim 22, wherein ARIA-H is detected by a brain MRI performed on the human subject after administering dose 6 of the antibody and prior to administering dose 7 of the antibody.

25. The method of claim 22, wherein a brain hemorrhage that is greater than 1 cm in diameter is detected during treatment with anti-amyloid-beta antibody and permanently discontinuing administration of anti-beta-amyloid antibody.

26. The method of any one of claims 22-25, further comprising administering further doses of the anti-beta-amyloid antibody to the human subject after a follow up MRI demonstrates radiographic stabilization.

27. The method of claim 26, wherein the amount of antibody administered in each of the further doses is the same as or greater than the amount of antibody administered in the last dose administered prior to detection of ARIA-H.

28. A method for treating Alzheimer's disease in a human subject in need thereof, the method comprising:
administering to the human subject multiple doses of an anti-beta-amyloid antibody, wherein the human subject develops a hypersensitivity reaction; and
discontinuing treatment upon the first observation of any signs or symptoms consistent with a hypersensitivity reaction.

29. The method of claim 28, wherein the hypersensitivity reaction comprises angioedema, urticaria, or angioedema and urticaria.

30. The method of claim 28 or 29, wherein further comprising administering a therapeutic agent for treating the hypersensitivity reaction.

31. The method of any one of the preceding claims, wherein the human subject has a confirmed amyloid beta pathology prior to administering.

32. A method for treating Alzheimer's disease, the method comprising:
administering to a population of human subject multiple doses of an anti-beta-amyloid antibody,
wherein in those human subjects that develop Amyloid Related Imaging Abnormality-H (ARIA-H) during treatment that is radiographically (i) mild ARIA-H with symptoms or (ii) moderate or severe ARIA-H with or without symptoms, suspending treatment until a follow-up MRI demonstrates radiographic stabilization and/or any symptoms resolve.

33. The method of claim 32, wherein the method comprises continuing administration in those subjects that do not develop ARIA-H, or develop radiographically mild ARIA-H without symptoms.

34. A method for treating Alzheimer's disease, comprising:
administering to a population of human subject multiple doses of an anti-beta-amyloid antibody,
wherein in those human subjects that develop Amyloid Related Imaging Abnormality-E (ARIA-E) during treatment that is radiographically moderate or severe ARIA-E, suspending treatment until a follow-up MRI demonstrates radiographic stabilization and/or any symptoms resolve.

35. The method of claim 34, wherein the method comprises continuing administration in those subjects that do not develop ARIA-E, or develop radiographically mild ARIA-E without symptoms or mild ARIA-E with mild symptoms.

36. The method of any one of claims 32-35, wherein the population of human subjects have a confirmed amyloid beta pathology prior to administering the anti-beta-amyloid antibody.

37. The method of any one of claims 32-35, wherein the method comprises:
obtaining a brain MRI for each of the subjects of the population prior to administering the anti-beta-amyloid antibody.

38. The method of any one of claims 32-37, wherein the multiple doses of the anti-beta-amyloid antibody comprise:
(i) dose 1 and dose 2 at 1 mg/kg,
(ii) dose 3 and dose 4 at 3 mg/kg,
(iii) dose 5 and dose 6 at 6 mg/kg, and
(iv) dose 7 and beyond at 10 mg/kg.

39. The method of claim 38, wherein the method further comprises:
performing a brain MRI on the human subjects of the population after administering multiple doses of the anti-beta-amyloid antibody, and prior to administering one or more of doses of the antibody selected from dose 5, dose 7, dose 9 and dose 12 of the antibody.

40. The method of claim 38, wherein the method further comprises:
performing a brain MRI on the human subjects of the population after administering multiple doses of the anti-beta-amyloid antibody, and prior to administering one or more of the first dose of 6 mg/kg, the first dose of 10 mg/kg, the third dose of 10 mg/kg, and the sixth dose of 10 mg/kg.

41. The method of claim 38 or 39, wherein the method further comprises:
performing a brain MRI on the human subjects of the population after administering multiple doses of the anti-beta-amyloid antibody, and prior to administering each of dose 5, dose 7, dose 9 and dose 12 of the antibody.

42. The method of claim 38 or 39, wherein the method further comprises:
performing a brain MRI on the human subjects of the population after administering multiple doses of the anti-beta-amyloid antibody, and prior to administering each of the first dose of 6 mg/kg, the first dose of 10 mg/kg, the third dose of 10 mg/kg, and the sixth dose of 10 mg/kg.

43. The method of any one of the preceding claims, wherein the anti-beta-amyloid antibody comprises a heavy chain variable region (VH) and a light chain variable region (VL),
wherein the VH comprises a first complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

44. The method of claim 43, wherein the VH comprises the amino acid sequence of SEQ ID NO:1 and the VL comprises the amino acid sequence of SEQ ID NO:2.

45. The method of claim 43, wherein the anti-beta-amyloid antibody comprises a heavy chain comprising the amino acid sequence of SEQ ID NO:10 and a light chain comprising the amino acid sequence of SEQ ID NO:11.

46. The method of any one of the preceding claims, wherein the anti-beta-amyloid antibody is administered as an intravenous infusion over approximate one hour via a 0.2 or 0.22 micron in-line filter.

47. The method of any one of the preceding claims, wherein the Alzheimer's disease is mild Alzheimer's disease, early Alzheimer's disease, prodromal Alzheimer's disease, mild Alzheimer's disease dementia, mild cognitive impairment due to Alzheimer's disease, mid-stage Alzheimer's disease, or late-stage Alzheimer's disease.

48. The method of any one of the preceding claims, further comprising measuring the levels of p-tau and/or t-tau in the CSF of the human subject prior to, during, and/or post treatment.

49. The method of any one of the preceding claims, wherein the anti-beta-amyloid antibody is formulated at a concentration of 50 mg/ml to 250 mg/ml in a pharmaceutical composition comprising:
Arg·HCl at a concentration of 50 mM to 200 mM;
methionine at a concentration of 5 mM to 20 mM;
a buffer at a concentration of 10 mM to 30 mM, wherein the buffer is selected from the group consisting of histidine, acetate, succinate, and citrate; and
PS80 at a concentration of 0.01% to 0.1%, and
wherein the composition has a pH of 5.2 to 6.0.

50. The method of any one of the preceding claims, wherein the anti-beta-amyloid antibody is formulated at a concentration of 100 mg/ml in a pharmaceutical composition comprising:
Arg·HCl at a concentration of 150 mM;
methionine at a concentration of 10 mM;
histidine at a concentration of 20 mM; and
PS80 at a concentration of 0.05%,
wherein the composition has a pH of 5.5.
